(19) United States
(12) Patent Application Publication
(10) Pub. No.: US 2024/0390024 A1
(43) Pub. Date: Nov. 28, 2024

(54) END EFFECTOR CONTROL AND CALIBRATION

(71) Applicant: Cilag GmbH International, Zug (CH)

(72) Inventors: Phillip H. Clauda, Cincinnati, OH (US); Cameron Nott, Fairfield, OH (US); John F. Cummings, Madeira, OH (US); David J. Cagle, San Francisco, CA (US); Daniel J. Ulrich, Hamilton, OH (US)

(21) Appl. No.: 18/670,238

(22) Filed: May 21, 2024

Related U.S. Application Data
(63) Continuation of application No. 17/665,163, filed on Feb. 4, 2022, now Pat. No. 11,998,230.
Application No. 17/665,163 is a continuation of application No. 15/363,244, filed on Nov. 29, 2016, now Pat. No. 11,266,430.

(51) Int. Cl. A61B 17/32; A61B 17/00; A61B 17/28; A61B 17/29; A61B 17/295; A61B 18/00; A61B 18/14

(52) U.S. Cl. CPC A61B 17/320092; A61B 17/2909; A61B 17/295; A61B 17/32; A61B 18/1445; A61B 2017/00026; A61B 2017/00039; A61B 2017/00128; A61B 2017/00725; A61B 2017/00977; A61B 2017/2808; A61B 2017/2925; A61B 2017/320094; A61B 2017/320095; A61B 2018/00595; A61B 2018/00607; A61B 2018/00642; A61B 2018/00666; A61B 2018/00702; A61B 2018/00875; A61B 2018/00994

(57) ABSTRACT

Methods and apparatus for end effector control and calibration are described. The method may include detecting a signal in response to movement of a first tube relative to a second tube, the first tube driving movement of a clamp arm of the end effector. The method may further include determining a clamp arm position of the end effector relative to a ultrasonic blade of the end effector based on the signal. The method may also include adjusting a power output to the ultrasonic blade of the end effector based on the clamp arm position.

### CROSS-REFERENCE TO RELATED APPLICATIONS

This application is a continuation application claiming priority under 35 U.S.C. § 120 to U.S. Patent Application Ser. No. 17/665,163, entitled END EFFECTOR CONTROL AND CALIBRATION, filed on Feb. 4, 2022, now U.S. Patent Application Publication No. 2022/0226014, which is a continuation application claiming priority under 35 U.S.C. § 120 to U.S. patent application Ser. No. 15/363,244, entitled END EFFECTOR CONTROL AND CALIBRATION, filed on Nov. 29, 2016, which issued on Mar. 8, 2022 as U.S. Pat. No. 11,266,430, the entire disclosure of which is hereby incorporated by reference herein.

### TECHNICAL FIELD

The technical field may generally relate to controlling surgical instruments, and in particular, controlling and calibrating end effectors of surgical instruments.

### BACKGROUND

Various aspects are directed to surgical instruments, and controlling and calibrating end effectors of surgical instruments.

For example, ultrasonic surgical devices are finding increasingly widespread applications in surgical procedures by virtue of their unique performance characteristics. Depending upon specific device configurations and operational parameters, ultrasonic surgical devices can provide substantially simultaneous transection of tissue and homeostasis by coagulation, desirably minimizing patient trauma. An ultrasonic surgical device may comprise a handpiece containing an ultrasonic transducer, and an instrument coupled to the ultrasonic transducer having a distally mounted end effector (e.g., an ultrasonic blade and a clamp arm, where the clamp arm may comprise a non-stick tissue pad) to cut and seal tissue. In some cases, the instrument may be permanently affixed to the handpiece. In other cases, the instrument may be detachable from the handpiece, as in the case of a disposable instrument or an instrument that is interchangeable between different handpieces. The end effector transmits ultrasonic energy to tissue brought into contact with the end effector to realize cutting and sealing action. Ultrasonic surgical devices of this nature can be configured for open surgical use, laparoscopic, or endoscopic surgical procedures including robotic-assisted procedures.

Ultrasonic energy cuts and coagulates tissue using temperatures lower than those used in electro surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied and the selected excursion level of the end effector.

### SUMMARY

In one aspect, a method for controlling an end effector may include detecting a signal in response to movement of a first tube relative to a second tube, the first tube driving movement of a clamp arm of the end effector. The method may also include determining a clamp arm position of the end effector relative to a ultrasonic blade of the end effector based on the signal. The method may additionally include adjusting a power output to the ultrasonic blade of the end effector based on the clamp arm position.

One or more of the following features may be included. The first tube may be an inner tube and the second tube may be an outer tube, the inner tube being moveable relative to the outer tube, the outer tube being static relative to the inner tube. The method may further include detecting the signal using a Hall-effect sensor and a magnet positioned on the first tube. The method may also include moving a magnet positioned on the first tube relative to a Hall-effect sensor as the first tube drives movement of the clamp arm of the end effector. The method may additionally include adjusting the power output to the ultrasonic blade of the end effector using an ultrasonic transducer based on a voltage change in a Hall-effect sensor. Moreover, the method may include adjusting the power output to the ultrasonic blade of the end effector dynamically, based on a travel ratio that changes as the clamp arm approaches the ultrasonic blade. Furthermore, the method may include adjusting the power output to the ultrasonic blade of the end effector dynamically, using a proportional-integral controller, based on a travel ratio that changes as the clamp arm approaches the ultrasonic blade.

In one or more implementations, the method may include determining a type of tissue between the clamp arm and the ultrasonic blade based on the signal. The method may also include adjusting the power output to the ultrasonic blade of the end effector based on the type of tissue. The method may additionally include, in response to determining that the type of tissue between the clamp and the ultrasonic blade is a small vessel, reducing the power output to the ultrasonic blade of the end effector by an amount less than for a large vessel. Moreover, the method may include in response to determining that the type of tissue between the clamp and the ultrasonic blade is a large vessel, reducing the power output to the ultrasonic blade of the end effector by an amount more than for a small vessel.

In one aspect, an apparatus for controlling an end effector may include a sensor configured to detect a signal in response to movement of a first tube relative to a second tube, the first tube driving movement of a clamp arm of the end effector. The apparatus may also include a processor configured to determine a clamp arm position of the end effector relative to a ultrasonic blade of the end effector based on the signal. The apparatus may further include a transducer configured to adjust a power output to the ultrasonic blade of the end effector based on the clamp arm position.

One or more of the following features may be included. The first tube may be an inner tube and the second tube may be an outer tube, the inner tube being moveable relative to the outer tube, the outer tube being static relative to the inner tube. The apparatus may further include a magnet positioned on the first tube wherein the sensor is a Hall-effect sensor used to detect the signal based on a position of the magnet. The magnet may be positioned on the first tube moves relative to a Hall-effect sensor as the first tube drives movement of the clamp arm of the end effector. The transducer may be an ultrasonic transducer configured to adjust the power output to the ultrasonic blade of the end effector based on a voltage change in a Hall-effect sensor. The apparatus may also include a proportional-integral controller configured to adjust the power output to the ultrasonic blade of the end effector dynamically, based on a travel ratio that changes as the clamp arm approaches the ultrasonic blade.

In one aspect, a method for calibrating an apparatus for controlling an end effector may include detecting a first signal corresponding to a fully open position of a clamp arm and a ultrasonic blade of the end effector. The method may also include detecting a second signal corresponding to an intermediate position of the clamp arm and the ultrasonic blade of the end effector, the intermediate position resulting from clamping a rigid body between the clamp arm and the ultrasonic blade. The method may additionally include detecting a third signal corresponding to a fully closed position of the clamp arm and the ultrasonic blade of the end effector. The method may further include determining a best fit curve to represent signal strength as a function of sensor displacement based on at least the first, second, and third signals, the fully open, intermediate, and fully closed positions, and a dimension of the rigid body. Moreover, the method may include creating a lookup table based on at least the first, second, and third signals, and the fully open, intermediate, and fully closed positions.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is an elevation view of an example surgical instrument in accordance with one aspect of the present disclosure

FIG. 2 is a perspective view of an example surgical instrument in accordance with one aspect of the present disclosure;

FIG. 3 illustrates an example end effector of a surgical instruments in accordance with one aspect of the present disclosure;

FIG. 4 illustrates an example end effector of a surgical instruments in accordance with one aspect of the present disclosure;

FIG. 5 is an exploded view of one aspect of a surgical instrument in accordance with one aspect of the present disclosure;

FIG. 6 illustrates a diagram of a surgical instrument in accordance with one aspect of the present disclosure;

FIG. 7 illustrates a structural view of a generator architecture in accordance with one aspect of the present disclosure;

FIGS. 8A-8C illustrate functional views of a generator architecture in accordance with one aspect of the present disclosure;

FIG. 9 illustrates a controller for monitoring input devices and controlling output devices in accordance with one aspect of the present disclosure;

FIGS. 10A and 10B illustrate structural and functional aspects of one aspect of the generator in accordance with one aspect the present disclosure;

FIG. 11 illustrates an example end effectors and shaft of a surgical instrument in accordance with one aspect of the present disclosure;

FIG. 12 illustrates an example Hall-effect sensor and magnet configuration in accordance with one aspect of the present disclosure where the Hall-effect sensor is fixed and the magnet moves in a line perpendicular to the face of the Hall sensor;

FIG. 13A illustrates an example Hall-effect sensor and magnet configuration in accordance with one aspect of the present disclosure where the Hall-effect sensor is fixed and the magnet moves in a line parallel to the face of the Hall-effect sensor;

FIG. 13B illustrates an example Hall-effect sensor and magnet configuration in accordance with one aspect of the present disclosure where the Hall-effect sensor is fixed and the magnet moves in a line parallel to the face of the Hall-effect sensor;

FIG. 14A is a table of output voltage of a Hall-effect sensor as a function of distance as a clamp arm moves from a fully closed position to a fully open position in accordance with one aspect the present disclosure;

FIG. 14B is a graph of output voltage of a Hall-effect sensor as a function of distance as a clamp arm moves from a fully closed position to a fully open position in accordance with one aspect the present disclosure;

FIG. 15A is a top view of a Hall-effect sensor and magnet configurations in a surgical instrument and corresponding open jaws end effector position in accordance with one aspect of the present disclosure;

FIG. 15B is a top view of a Hall-effect sensor and magnet configurations in a surgical instrument and corresponding closed jaws end effector position in accordance with one aspect of the present disclosure;

FIG. 16 illustrates a plan view of a system comprising a Hall-effect sensor and magnet configuration in a surgical instrument in accordance with one aspect of the present disclosure;

FIG. 17A illustrates a view of an Hall-effect sensor and magnet configurations in the context of a surgical instrument in accordance with one aspect of the present disclosure;

FIG. 17B illustrates a view of an Hall-effect sensor and magnet configurations in the context of a surgical instrument in accordance with one aspect of the present disclosure;

FIG. 18 illustrates a Hall-effect sensor and magnet configuration in a surgical instrument in accordance with one aspect of the present disclosure;

FIG. 19A illustrates a Hall-effect sensor and magnet configuration in accordance with one aspect of the present disclosure;

FIG. 19B illustrates Hall-effect sensor and magnet configurations in a surgical instrument in accordance with one aspect of the present disclosure;

FIG. 20 is a graph of a curve depicting Travel Ratio (TR) along the y-axis, based on Hall-effect sensor output voltage, as a function of Time (Sec) along the x-axis in accordance with one aspect of the present disclosure;

FIG. 21 illustrates a graph of a first curve depicting Travel Ratio (TR) along the left y-axis, based on Hall-effect sensor output voltage, as a function of Time (Sec) along the x-axis in accordance with one aspect of the present disclosure;

FIG. 22 illustrates charts showing Proportional-Integral control of power output to an ultrasonic blade in accordance with one aspect of the present disclosure;

FIG. 23 illustrates several vessels that were sealed using the techniques and features described herein in accordance with one aspect of the present disclosure;

FIG. 24 illustrates a graph of a best fit curve of Hall-effect sensor output voltage as a function of distance for various positions of the clamp arm as the clamp arm moves between fully closed to a fully open positions in accordance with one aspect of the present disclosure;

FIGS. 25-28 illustrate an end effector being calibrated in four different configurations in accordance with various aspects of the present disclosure using gage pins for two of the configurations for recording a Hall-effect sensor response corresponding to various positions of the clamp arm to record four data points (1-4) to create a best fit curve during production, where:

FIG. 25 illustrates an end effector in a fully open configuration to record a first data point (1) in accordance with one aspect of the present disclosure;

FIG. 26 illustrates an end effector in a second intermediate configuration grasping a first gage pin of a known diameter to record a second data point (2) in accordance with one aspect of the present disclosure;

FIG. 27 illustrates an end effector in a third intermediate configuration grasping a second gage pin of a known diameter to record a third data point (3) in accordance with one aspect of the present disclosure; and

FIG. 28 illustrates an end effector in a fully closed configuration to record a fourth data point (4) in accordance with one aspect of the present disclosure;

FIGS. 29A-D illustrate an example surgical instrument in accordance with one aspect of the present disclosure and charts showing example output power level in a hemostasis mode for small and large vessels, where:

FIG. 29A is a schematic diagram of surgical instrument configured to seal small and large vessels in accordance with one aspect of the present disclosure;

FIG. 29B is a diagram of an example range of a small vessel and a large vessel and the relative position of a clamp arm of the end effector in accordance with one aspect of the present disclosure;

FIG. 29C is a graph that depicts a process for sealing small vessels by applying various ultrasonic energy levels for a different periods of time in accordance with one aspect of the present disclosure; and

FIG. 29D is a graph that depicts a process for sealing large vessels by applying various ultrasonic energy levels for different periods of time in accordance with one aspect of the present disclosure;

FIG. 30 is a logic diagram illustrating an example process for determining whether hemostasis mode should be used, in accordance with one aspect of the present disclosure;

FIG. 31 is a logic diagram illustrating an example process for end effector control in accordance with one aspect of the present disclosure;

FIG. 32 is a logic diagram illustrating an example process for calibrating an apparatus for controlling for an end effector in accordance with one aspect of the present disclosure;

FIG. 33 is a logic diagram of a process for tracking wear of the tissue pad portion of the clamp arm and compensating for resulting drift of the Hall-effect sensor and determining tissue coefficient of friction in accordance with one aspect of the present disclosure;

FIG. 34 illustrates a Hall-effect sensor system that can be employed with the process of FIG. 33 in accordance with one aspect of the present disclosure; and

FIG. 35 illustrates one aspect of a ramp type counter analog-to-digital converter (ADC) that may be employed with the Hall-effect sensor system of FIG. 34 in accordance with one aspect of the present disclosure.

### DESCRIPTION

Various aspects described herein are directed to surgical instruments comprising distally positioned, articulatable jaw assemblies. The jaw assemblies may be utilized in lieu of or in addition to shaft articulation. For example, the jaw assemblies may be utilized to grasp tissue and move it towards an ultrasonic blade, RF electrodes or other component for treating tissue.

In one aspect, a surgical instrument may comprise an end effector with an ultrasonic blade extending distally therefrom. The jaw assembly may be articulatable and may pivot about at least two axes. A first axis, or wrist pivot axis, may be substantially perpendicular to a longitudinal axis of the instrument shaft. The jaw assembly may pivot about the wrist pivot axis from a first position where the jaw assembly is substantially parallel to the ultrasonic blade to a second position where the jaw assembly is not substantially parallel to the ultrasonic blade. In addition, the jaw assembly may comprise first and second jaw members that are pivotable about a second axis or jaw pivot axis. The jaw pivot axis may be substantially perpendicular to the wrist pivot axis. In some aspects, the jaw pivot axis itself may pivot as the jaw assembly pivots about the wrist pivot axis. The first and second jaw members may be pivotably relative to one another about the jaw pivot axis such that the first and second jaw members may “open” and “close.” Additionally, in some aspects, the first and second jaw members are also pivotable about the jaw pivot axis together such that the direction of the first and second jaw members may change.

Reference will now be made in detail to several aspects, including aspects showing example implementations of manual and robotic surgical instruments with end effectors comprising ultrasonic and/or electro surgical elements. Wherever practicable similar or like reference numbers may be used in the figures and may indicate similar or like functionality. The figures depict example aspects of the disclosed surgical instruments and/or methods of use for purposes of illustration only. One skilled in the art will readily recognize from the following description that alternative example aspects of the structures and methods illustrated herein may be employed without departing from the principles described herein.

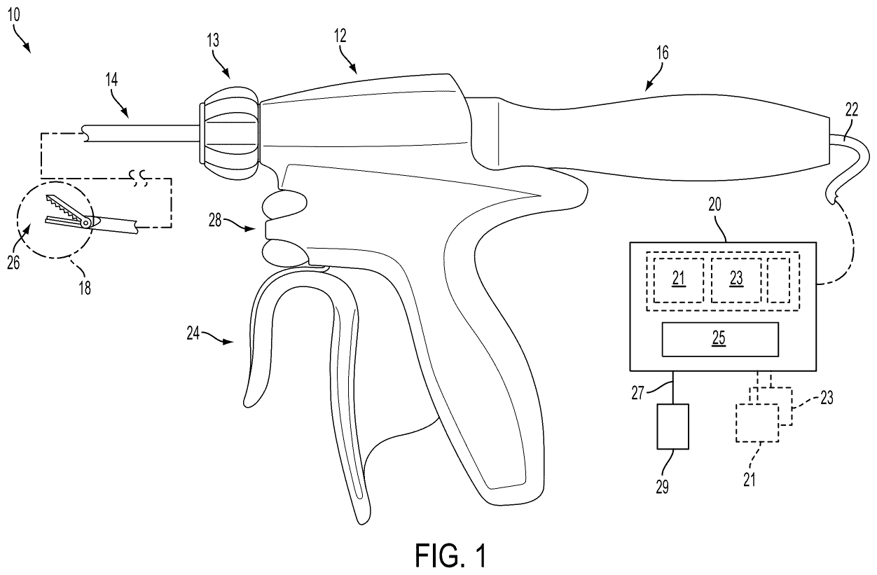

FIG. 1 is a right side view of one aspect of an ultrasonic surgical instrument 10. In the illustrated aspect, the ultrasonic surgical instrument 10 may be employed in various surgical procedures including endoscopic or traditional open surgical procedures. In one aspect, the ultrasonic surgical instrument 10 comprises a handle assembly 12, an elongated shaft assembly 14, and an ultrasonic transducer 16. The handle assembly 12 comprises a trigger assembly 24, a distal rotation assembly 13, and a switch assembly 28. The elongated shaft assembly 14 comprises an end effector assembly 26, which comprises elements to dissect tissue or mutually grasp, cut, and coagulate vessels and/or tissue, and actuating elements to actuate the end effector assembly 26. The handle assembly 12 is adapted to receive the ultrasonic transducer 16 at the proximal end. The ultrasonic transducer 16 is mechanically engaged to the elongated shaft assembly 14 and portions of the end effector assembly 26. The ultrasonic transducer 16 is electrically coupled to a generator 20 via a cable 22. Although the majority of the drawings depict a multiple end effector assembly 26 for use in connection with laparoscopic surgical procedures, the ultrasonic surgical instrument 10 may be employed in more traditional open surgical procedures and in other aspects, may be configured for use in endoscopic procedures. For the purposes herein, the ultrasonic surgical instrument 10 is described in terms of an endoscopic instrument; however, it is contemplated that an open and/or laparoscopic version of the ultrasonic surgical instrument 10 also may include the same or similar operating components and features as described herein.

In various aspects, the generator 20 comprises several functional elements, such as modules and/or blocks. Different functional elements or modules may be configured for driving different kinds of surgical devices. For example, an ultrasonic generator module 21 may drive an ultrasonic device, such as the ultrasonic surgical instrument 10. In some example aspects, the generator 20 also comprises an electrosurgery/RF generator module 23 for driving an electrosurgical device (or an electro surgical aspect of the ultrasonic surgical instrument 10). In the example aspect illustrated in FIG. 1, the generator 20 includes a control system 25 integral with the generator 20, and a foot switch 29 connected to the generator via a cable 27. The generator 20 may also comprise a triggering mechanism for activating a surgical instrument, such as the instrument 10. The triggering mechanism may include a power switch (not shown) as well as a foot switch 29. When activated by the foot switch 29, the generator 20 may provide energy to drive the acoustic assembly of the surgical instrument 10 and to drive the end effector 18 at a predetermined excursion level. The generator 20 drives or excites the acoustic assembly at any suitable resonant frequency of the acoustic assembly and/or derives the therapeutic/sub-therapeutic electromagnetic/RF energy. In one aspect, the electrosurgical/RF generator module 23 may be implemented as an electrosurgery unit (ESU) capable of supplying power sufficient to perform bipolar electrosurgery using radio frequency (RF) energy. In one aspect, the ESU can be a bipolar ERBE ICC 350 sold by ERBE USA, Inc. of Marietta, Ga. In bipolar electrosurgery applications, as previously discussed, a surgical instrument having an active electrode and a return electrode can be utilized, wherein the active electrode and the return electrode can be positioned against, or adjacent to, the tissue to be treated such that current can flow from the active electrode to the return electrode through the tissue. Accordingly, the electrosurgical/RF module 23 generator may be configured for therapeutic purposes by applying electrical energy to the tissue T sufficient for treating the tissue (e.g., cauterization). For example, in some aspects, the active and/or return electrode may be positioned on the jaw assembly described herein.

In one aspect, the electrosurgical/RF generator module 23 may be configured to deliver a subtherapeutic RF signal to implement a tissue impedance measurement module. In one aspect, the electrosurgical/RF generator module 23 comprises a bipolar radio frequency generator. In one aspect, the electrosurgical/RF generator module 23 may be configured to monitor electrical impedance Z, of tissue T and to control the characteristics of time and power level based on the tissue T by way of a return electrode provided on a clamp member of the end effector assembly 26. Accordingly, the electrosurgical/RF generator module 23 may be configured for subtherapeutic purposes for measuring the impedance or other electrical characteristics of the tissue T. Techniques and circuit configurations for measuring the impedance or other electrical characteristics of tissue Tare discussed in more detail in commonly assigned U.S. Patent Publication No. 2011/0015631, titled “Electrosurgical Generator for Ultrasonic Surgical Instrument,” the disclosure of which is herein incorporated by reference in its entirety.

A suitable ultrasonic generator module 21 may be configured to functionally operate in a manner similar to the GEN300 sold by Ethicon Endo-Surgery, Inc. of Cincinnati, Ohio as is disclosed in one or more of the following U.S. patents, all of which are incorporated by reference herein: U.S. Pat. No. 6,480,796 (Method for Improving the Start Up of an Ultrasonic System Under Zero Load Conditions); U.S. Pat. No. 6,537,291 (Method for Detecting a Loose Blade in a Hand Piece Connected to an Ultrasonic Surgical System); U.S. Pat. No. 6,662,127 (Method for Detecting Presence of a Blade in an Ultrasonic System); U.S. Pat. No. 6,977,495 (Detection Circuitry for Surgical Handpiece System); U.S. Pat. No. 7,077,853 (Method for Calculating Transducer Capacitance to Determine Transducer Temperature); U.S. Pat. No. 7,179,271 (Method for Driving an Ultrasonic System to Improve Acquisition of Ultrasonic Blade Resonance Frequency at Startup); and U.S. Pat. No. 7,273,483 (Apparatus and Method for Alerting Generator Function in an Ultrasonic Surgical System).

It will be appreciated that in various aspects, the generator 20 may be configured to operate in several modes. In one mode, the generator 20 may be configured such that the ultrasonic generator module 21 and the electrosurgical/RF generator module 23 may be operated independently.

For example, the ultrasonic generator module 21 may be activated to apply ultrasonic energy to the end effector assembly 26 and subsequently, either therapeutic sub-therapeutic RF energy may be applied to the end effector assembly 26 by the electrosurgical/RF generator module 23. As previously discussed, the sub-therapeutic electrosurgical/RF energy may be applied to tissue clamped between claim elements of the end effector assembly 26 to measure tissue impedance to control the activation, or modify the activation, of the ultrasonic generator module 21. Tissue impedance feedback from the application of the sub-therapeutic energy also may be employed to activate a therapeutic level of the electrosurgical/RF generator module 23 to seal the tissue (e.g., vessel) clamped between claim elements of the end effector assembly 26.

In another aspect, the ultrasonic generator module 21 and the electrosurgical/RF generator module 23 may be activated simultaneously. In one example, the ultrasonic generator module 21 is simultaneously activated with a sub-therapeutic RF energy level to measure tissue impedance simultaneously while the ultrasonic blade of the end effector assembly 26 cuts and coagulates the tissue (or vessel) clamped between the clamp elements of the end effector assembly 26. Such feedback may be employed, for example, to modify the drive output of the ultrasonic generator module 21. In another example, the ultrasonic generator module 21 may be driven simultaneously with electrosurgical/RF generator module 23 such that the ultrasonic blade portion of the end effector assembly 26 is employed for cutting the damaged tissue while the electrosurgical/RF energy is applied to electrode portions of the end effector clamp assembly 26 for sealing the tissue (or vessel).

When the generator 20 is activated via the triggering mechanism, electrical energy is continuously applied by the generator 20 to a transducer stack or assembly of the acoustic assembly. In another aspect, electrical energy is intermittently applied (e.g., pulsed) by the generator 20. A phaselocked loop in the control system of the generator 20 may monitor feedback from the acoustic assembly. The phase lock loop adjusts the frequency of the electrical energy sent by the generator 20 to match the resonant frequency of the selected longitudinal mode of vibration of the acoustic assembly. In addition, a second feedback loop in the control system 25 maintains the electrical current supplied to the acoustic assembly at a pre-selected constant level in order to achieve substantially constant excursion at the end effector 18 of the acoustic assembly. In yet another aspect, a third feedback loop in the control system 25 monitors impedance between electrodes located in the end effector assembly 26. Although FIGS. 1-5 show a manually operated ultrasonic surgical instrument, it will be appreciated that ultrasonic surgical instruments may also be used in robotic applications, for example, as described herein as well as combinations of manual and robotic applications.

In ultrasonic operation mode, the electrical signal supplied to the acoustic assembly may cause the distal end of the end effector 18, to vibrate longitudinally in the range of, for example, approximately 20 kHz to 250 kHz. According to various aspects, the ultrasonic blade 22 may vibrate in the range of about 54 kHz to 56 kHz, for example, at about 55.5 kHz. In other aspects, the ultrasonic blade 22 may vibrate at other frequencies including, for example, about 31 kHz or about 80 kHz. The excursion of the vibrations at the ultrasonic blade can be controlled by, for example, controlling the amplitude of the electrical signal applied to the transducer assembly of the acoustic assembly by the generator 20. As noted above, the triggering mechanism of the generator 20 allows a user to activate the generator 20 so that electrical energy may be continuously or intermittently supplied to the acoustic assembly. The generator 20 also has a power line for insertion in an electro-surgical unit or conventional electrical outlet. It is contemplated that the generator 20 can also be powered by a direct current (DC) source, such as a battery. The generator 20 can comprise any suitable generator, such as Model No. GEN04, and/or Model No. GEN11 available from Ethicon Endo-Surgery, Inc.

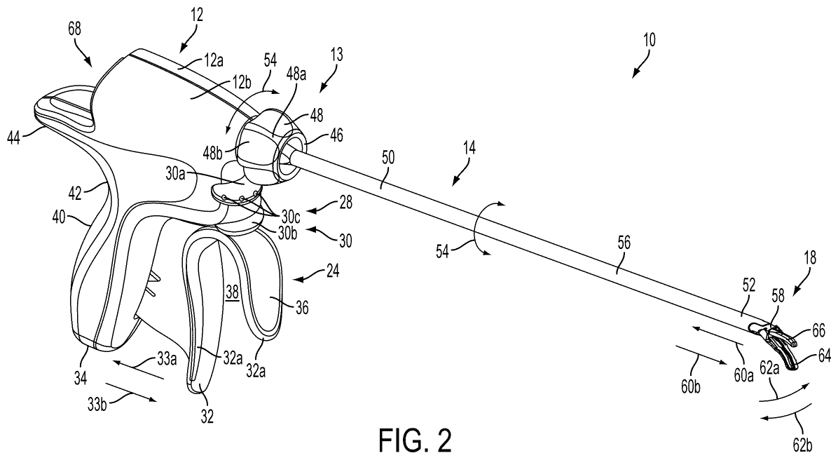

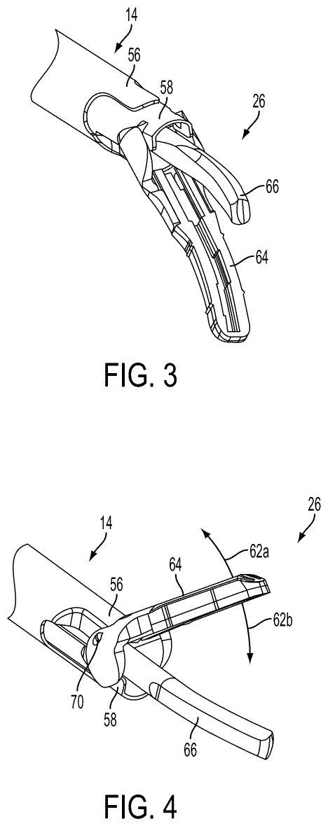

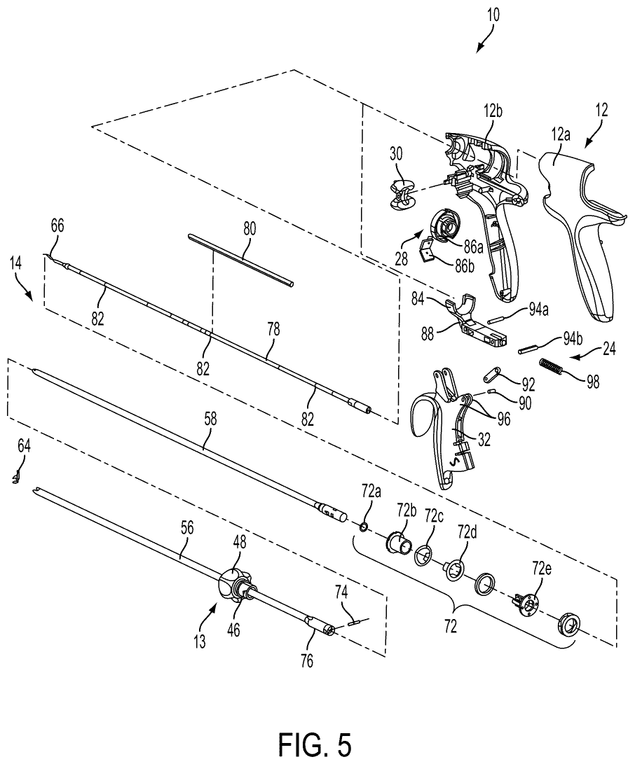

FIG. 2 is a left perspective view of one example aspect of the ultrasonic surgical instrument 10 showing the handle assembly 12, the distal rotation assembly 13, and the elongated shaft assembly 14. FIG. 3 shows the end effector assembly 26. In the illustrated aspect the elongated shaft assembly 14 comprises a distal end 52 dimensioned to mechanically engage the end effector assembly 26 and a proximal end 50 that mechanically engages the handle assembly 12 and the distal rotation assembly 13. The proximal end 50 of the elongated shaft assembly 14 is received within the handle assembly 12 and the distal rotation assembly 13. More details relating to the connections between the elongated shaft assembly 14, the handle assembly 12, and the distal rotation assembly 13 are provided in the description of FIG. 5. In the illustrated aspect, the trigger assembly 24 comprises a trigger 32 that operates in conjunction with a fixed handle 34. The fixed handle 34 and the trigger 32 are ergonomically formed and adapted to interface comfortably with the user. The fixed handle 34 is integrally associated with the handle assembly 12. The trigger 32 is pivotally movable relative to the fixed handle 34 as explained in more detail below with respect to the operation of the ultrasonic surgical instrument 10. The trigger 32 is pivotally movable in direction 33a toward the fixed handle 34 when the user applies a squeezing force against the trigger 32. A spring element 98 (FIG. 5) causes the trigger 32 to pivotally move in direction 33b when the user releases the squeezing force against the trigger 32.

In one example aspect, the trigger 32 comprises an elongated trigger hook 36, which defines an aperture 38 between the elongated trigger hook 36 and the trigger 32. The aperture 38 is suitably sized to receive one or multiple fingers of the user therethrough. The trigger 32 also may comprise a resilient portion 32a molded over the trigger 32 substrate. The overmolded resilient portion 32a is formed to provide a more comfortable contact surface for control of the trigger 32 in outward direction 33b. In one example aspect, the overmolded resilient portion 32a may be provided over a portion of the elongated trigger hook 3 6. The proximal surface of the elongated trigger hook 32 remains uncoated or coated with a non-resilient substrate to enable the user to easily slide their fingers in and out of the aperture 38. In another aspect, the geometry of the trigger forms a fully closed loop which defines an aperture suitably sized to receive one or multiple fingers of the user therethrough. The fully closed loop trigger also may comprise a resilient portion molded over the trigger substrate.

In one example aspect, the fixed handle 34 comprises a proximal contact surface 40 and a grip anchor or saddle surface 42. The saddle surface 42 rests on the web where the thumb and the index finger are joined on the hand. The proximal contact surface 40 has a pistol grip contour that receives the palm of the hand in a normal pistol grip with no rings or apertures. The profile curve of the proximal contact surface 40 may be contoured to accommodate or receive the palm of the hand. A stabilization tail 44 is located towards a more proximal portion of the handle assembly 12. The stabilization tail 44 may be in contact with the uppermost web portion of the hand located between the thumb and the index finger to stabilize the handle assembly 12 and make the handle assembly 12 more controllable.

In one example aspect, the switch assembly 28 may comprise a toggle switch 30. The toggle switch 30 may be implemented as a single component with a central pivot 304 located within inside the handle assembly 12 to eliminate the possibility of simultaneous activation. In one example aspect, the toggle switch 30 comprises a first projecting knob 30a and a second projecting knob 30b to set the power setting of the ultrasonic transducer 16 between a minimum power level (e.g., MIN) and a maximum power level (e.g., MAX). In another aspect, the rocker switch may pivot between a standard setting and a special setting. The special setting may allow one or more special programs, processes, or algorithms and described herein to be implemented by the device. The toggle switch 30 rotates about the central pivot as the first projecting knob 30a and the second projecting knob 30b are actuated The one or more projecting knobs 30a, 30b are coupled to one or more arms that move through a small arc and cause electrical contacts to close or open an electric circuit to electrically energize or de-energize the ultrasonic transducer 16 in accordance with the activation of the first or second projecting knobs 30a, 30b. The toggle switch 30 is coupled to the generator 20 to control the activation of the ultrasonic transducer 16. The toggle switch 30 comprises one or more electrical power setting switches to activate the ultrasonic transducer 16 to set one or more power settings for the ultrasonic transducer 16. The forces required to activate the toggle switch 30 are directed substantially toward the saddle point 42, thus avoiding any tendency of the instrument to rotate in the hand when the toggle switch 30 is activated.

In one example aspect, the first and second projecting knobs 30a, 30b are located on the distal end of the handle assembly 12 such that they can be easily accessible by the user to activate the power with minimal, or substantially no, repositioning of the hand grip, making it suitable to maintain control and keep attention focused on the surgical site (e.g., a monitor in a laparoscopic procedure) while activating the toggle switch 30. The projecting knobs 30a, 30b may be configured to wrap around the side of the handle assembly 12 to some extent to be more easily accessible by variable finger lengths and to allow greater freedom of access to activation in awkward positions or for shorter fingers. In the illustrated aspect, the first projecting knob 30a comprises a plurality of tactile elements 30c, e.g., textured projections or “bumps” in the illustrated aspect, to allow the user to differentiate the first projecting knob 30a from the second projecting knob 30b. It will be appreciated by those skilled in the art that several ergonomic features may be incorporated into the handle assembly 12. Such ergonomic features are described in U.S. Pat. App. Pub. No. 2009/0105750 entitled “Ergonomic Surgical Instruments” which is incorporated by reference herein in its entirety.

In one example aspect, the toggle switch 30 may be operated by the hand of the user. The user may easily access the first and second projecting knobs 30a, 30b at any point while also avoiding inadvertent or unintentional activation at any time. The toggle switch 30 may readily be operated with a finger to control the power to the ultrasonic assembly 16 and/or to the ultrasonic assembly 16. For example, the index finger may be employed to activate the first contact portion 30a to turn on the ultrasonic assembly 16 to a maximum (MAX) power level. The index finger may be employed to activate the second contact portion 30b to turn on the ultrasonic assembly 16 to a minimum (MIN) power level. In another aspect, the rocker switch may pivot the instrument 10 between a standard setting and a special setting. The special setting may allow one or more special programs to be implemented by the instrument 10. The toggle switch 30 may be operated without the user having to look at the first or second projecting knob 30a, 30b. For example, the first projecting knob 30a or the second projecting knob 30b may comprise a texture or projections to tactilely differentiate between the first and second projecting knobs 30a, 30b without looking.

In one example aspect, the distal rotation assembly 13 is rotatable without limitation in either direction about a longitudinal axis “T.” The distal rotation assembly 13 is mechanically engaged to the elongated shaft assembly 14. The distal rotation assembly 13 is located on a distal end of the handle assembly 12. The distal rotation assembly 13 comprises a cylindrical hub 46 and a rotation knob 48 formed over the hub 46. The hub 46 mechanically engages the elongated shaft assembly 14. The rotation knob 48 may comprise fluted polymeric features and may be engaged by a finger (e.g., an index finger) to rotate the elongated shaft assembly 14. The hub 46 may comprise a material molded over the primary structure to form the rotation knob 48. The rotation knob 48 may be overmolded over the hub 46. The hub 46 comprises an end cap portion 46a that is exposed at the distal end. The end cap portion 46a of the hub 46 may contact the surface of a trocar during laparoscopic procedures. The hub 46 may be formed of a hard durable plastic such as polycarbonate to alleviate any friction that may occur between the end cap portion 46a and the trocar. The rotation knob 48 may comprise “scallops” or flutes formed of raised ribs 48a and concave portions 48b located between the ribs 48a to provide a more precise rotational grip. In one example aspect, the rotation knob 48 may comprise a plurality of flutes (e.g., three or more flutes). In other aspects, any suitable number of flutes may be employed. The rotation knob 48 may be formed of a softer polymeric material overmolded onto the hard plastic material. For example, the rotation knob 48 may be formed of pliable, resilient, flexible polymeric materials including Versaflex® TPE alloys made by GLS Corporation, for example. This softer overmolded material may provide a greater grip and more precise control of the movement of the rotation knob 48. It will be appreciated that any materials that provide adequate resistance to sterilization, are biocompatible, and provide adequate frictional resistance to surgical gloves may be employed to form the rotation knob 48.

In one example aspect, the handle assembly 12 is formed from two (2) housing portions or shrouds comprising a first portion 12a and a second portion 12b. From the perspective of a user viewing the handle assembly 12 from the distal end towards the proximal end, the first portion 12a is considered the right portion and the second portion 12b is considered the left portion. Each of the first and second portions 12a, 12b includes a plurality of interfaces 69 (FIG. 5) dimensioned to mechanically align and engage each another to form the handle assembly 12 and enclosing the internal working components thereof. The fixed handle 34, which is integrally associated with the handle assembly 12, takes shape upon the assembly of the first and second portions 12a and 12b of the handle assembly 12. A plurality of additional interfaces (not shown) may be disposed at various points around the periphery of the first and second portions 12a and 12b of the handle assembly 12 for ultrasonic welding purposes, e.g., energy direction/deflection points. The first and second portions 12a and 12b (as well as the other components described below) may be assembled together in any fashion known in the art. For example, alignment pins, snap-like interfaces, tongue and groove interfaces, locking tabs, adhesive ports, may all be utilized either alone or in combination for assembly purposes.

In one example aspect, the elongated shaft assembly 14 comprises a proximal end 50 adapted to mechanically engage the handle assembly 12 and the distal rotation assembly 13; and a distal end 52 adapted to mechanically engage the end effector assembly 26. The elongated shaft assembly 14 comprises an outer tubular sheath 56 and a reciprocating tubular actuating member 58 located within the outer tubular sheath 56. The proximal end of the tubular reciprocating tubular actuating member 58 is mechanically engaged to the trigger 32 of the handle assembly 12 to move in either direction 60A or 60B in response to the actuation and/or release of the trigger 32. The pivotably moveable trigger 32 may generate reciprocating motion along the longitudinal axis “T.” Such motion may be used, for example, to actuate the jaws or clamping mechanism of the end effector assembly 26. A series of linkages translate the pivotal rotation of the trigger 32 to axial movement of a yoke coupled to an actuation mechanism, which controls the opening and closing of the jaws of the clamping mechanism of the end effector assembly 26. The distal end of the tubular reciprocating tubular actuating member 58 is mechanically engaged to the end effector assembly 26. In the illustrated aspect, the distal end of the tubular reciprocating tubular actuating member 58 is mechanically engaged to a clamp arm assembly 64, which is pivotable about a pivot point 70 (FIG. 4), to open and close the clamp arm assembly 64 in response to the actuation and/or release of the trigger 32. For example, in the illustrated aspect, the clamp arm assembly 64 is movable in direction 62A from an open position to a closed position about a pivot point 70 when the trigger 32 is squeezed in direction 33a. The clamp arm assembly 64 is movable in direction 62B from a closed position to an open position about the pivot point 70 when the trigger 32 is released or outwardly contacted in direction 33b.

In one example aspect, the end effector assembly 26 is attached at the distal end 52 of the elongated shaft assembly 14 and includes a clamp arm assembly 64 and a ultrasonic blade 66. The jaws of the clamping mechanism of the end effector assembly 26 are formed by clamp arm assembly 64 and the ultrasonic blade 66. The ultrasonic blade 66 is ultrasonically actuatable and is acoustically coupled to the ultrasonic transducer 16. The trigger 32 on the handle assembly 12 is ultimately connected to a drive assembly, which together, mechanically cooperate to effect movement of the clamp arm assembly 64. Squeezing the trigger 32 in direction 33a moves the clamp arm assembly 64 in direction 62A from an open position, wherein the clamp arm assembly 64 and the ultrasonic blade 66 are disposed in a spaced relation relative to one another, to a clamped or closed position, wherein the clamp arm assembly 64 and the ultrasonic blade 66 cooperate to grasp tissue therebetween. The clamp arm assembly 64 may comprise a clamp pad 69 to engage tissue between the ultrasonic blade 66 and the clamp arm 64. Releasing the trigger 32 in direction 33b moves the clamp arm assembly 64 in direction 62B from a closed relationship, to an open position, wherein the clamp arm assembly 64 and the ultrasonic blade 66 are disposed in a spaced relation relative to one another.

The proximal portion of the handle assembly 12 comprises a proximal opening 68 to receive the distal end of the ultrasonic assembly 16. The ultrasonic assembly 16 is inserted in the proximal opening 68 and is mechanically engaged to the elongated shaft assembly 14.

In one example aspect, the elongated trigger hook 36 portion of the trigger 32 provides a longer trigger lever with a shorter span and rotation travel. The longer lever of the elongated trigger hook 36 allows the user to employ multiple fingers within the aperture 38 to operate the elongated trigger hook 36 and cause the trigger 32 to pivot in direction 33b to open the jaws of the end effector assembly 26. For example, the user may insert three fingers (e.g., the middle, ring, and little fingers) in the aperture 38. Multiple fingers allows the surgeon to exert higher input forces on the trigger 32 and the elongated trigger hook 326 to activate the end effector assembly 26. The shorter span and rotation travel creates a more comfortable grip when closing or squeezing the trigger 32 in direction 33a or when opening the trigger 32 in the outward opening motion in direction 33b lessening the need to extend the fingers further outward. This substantially lessens hand fatigue and strain associated with the outward opening motion of the trigger 32 in direction 33b. The outward opening motion of the trigger may be spring-assisted by spring element 98 (FIG. 5) to help alleviate fatigue. The opening spring force is sufficient to assist the ease of opening, but not strong enough to adversely impact the tactile feedback of tissue tension during spreading dissection.

For example, during a surgical procedure either the index finger may be used to control the rotation of the elongated shaft assembly 14 to locate the jaws of the end effector assembly 26 in a suitable orientation. The middle and/or the other lower fingers may be used to squeeze the trigger 32 and grasp tissue within the jaws. Once the jaws are located in the desired position and the jaws are clamped against the tissue, the index finger can be used to activate the toggle switch 30 to adjust the power level of the ultrasonic transducer 16 to treat the tissue. Once the tissue has been treated, the user then may release the trigger 32 by pushing outwardly in the distal direction against the elongated trigger hook 36 with the middle and/or lower fingers to open the jaws of the end effector assembly 26. This basic procedure may be performed without the user having to adjust their grip of the handle assembly 12.

FIGS. 3-4 illustrate the connection of the elongated shaft assembly 14 relative to the end effector assembly 26. As previously described, in the illustrated aspect, the end effector assembly 26 comprises a clamp arm assembly 64 and a ultrasonic blade 66 to form the jaws of the clamping mechanism. The ultrasonic blade 66 may be an ultrasonically actuatable ultrasonic blade acoustically coupled to the ultrasonic transducer 16. The trigger 32 is mechanically connected to a drive assembly. Together, the trigger 32 and the drive assembly mechanically cooperate to move the clamp arm assembly 64 to an open position in direction 62A wherein the clamp arm assembly 64 and the ultrasonic blade 66 are disposed in spaced relation relative to one another, to a clamped or closed position in direction 62B wherein the clamp arm assembly 64 and the ultrasonic blade 66 cooperate to grasp tissue therebetween. The clamp arm assembly 64 may comprise a clamp pad 69 to engage tissue between the ultrasonic blade 66 and the clamp arm 64. The distal end of the tubular reciprocating tubular actuating member 58 is mechanically engaged to the end effector assembly 26. In the illustrated aspect, the distal end of the tubular reciprocating tubular actuating member 58 is mechanically engaged to the clamp arm assembly 64, which is pivotable about the pivot point 70, to open and close the clamp arm assembly 64 in response to the actuation and/or release of the trigger 32. For example, in the illustrated aspect, the clamp arm assembly 64 is movable from an open position to a closed position in direction 62B about a pivot point 70 when the trigger 32 is squeezed in direction 33a. The clamp arm assembly 64 is movable from a closed position to an open position in direction 62A about the pivot point 70 when the trigger 32 is released or outwardly contacted in direction 33b.

As previously discussed, the clamp arm assembly 64 may comprise electrodes electrically coupled to the electrosurgical/RF generator module 23 to receive therapeutic and/or sub-therapeutic energy, where the electrosurgical/RF energy may be applied to the electrodes either simultaneously or non simultaneously with the ultrasonic energy being applied to the ultrasonic blade 66. Such energy activations may be applied in any suitable combinations to achieve a desired tissue effect in cooperation with an algorithm or other control logic.

FIG. 5 is an exploded view of the ultrasonic surgical instrument 10 shown in FIG. 2. In the illustrated aspect, the exploded view shows the internal elements of the handle assembly 12, the handle assembly 12, the distal rotation assembly 13, the switch assembly 28, and the elongated shaft assembly 14. In the illustrated aspect, the first and second portions 12a, 12b mate to form the handle assembly 12. The first and second portions 12a, 12b each comprises a plurality of interfaces 69 dimensioned to mechanically align and engage one another to form the handle assembly 12 and enclose the internal working components of the ultrasonic surgical instrument 10. The rotation knob 48 is mechanically engaged to the outer tubular sheath 56 so that it may be rotated in circular direction 54 up to 360°. The outer tubular sheath 56 is located over the reciprocating tubular actuating member 58, which is mechanically engaged to and retained within the handle assembly 12 via a plurality of coupling elements 72. The coupling elements 72 may comprise an O-ring 72a, a tube collar cap 72b, a distal washer 72c, a proximal washer 72d, and a thread tube collar 72e. The reciprocating tubular actuating member 58 is located within a reciprocating yoke 84, which is retained between the first and second portions 12a, 12b of the handle assembly 12. The yoke 84 is part of a reciprocating yoke assembly 88. A series of linkages translate the pivotal rotation of the elongated trigger hook 32 to the axial movement of the reciprocating yoke 84, which controls the opening and closing of the jaws of the clamping mechanism of the end effector assembly 26 at the distal end of the ultrasonic surgical instrument 10. In one example aspect, a four-link design provides mechanical advantage in a relatively short rotation span, for example.

In one example aspect, an ultrasonic transmission waveguide 78 is disposed inside the reciprocating tubular actuating member 58. The distal end 52 of the ultrasonic transmission waveguide 78 is acoustically coupled (e.g., directly or indirectly mechanically coupled) to the ultrasonic blade 66 and the proximal end 50 of the ultrasonic transmission waveguide 78 is received within the handle assembly 12. The proximal end 50 of the ultrasonic transmission waveguide 78 is adapted to acoustically couple to the distal end of the ultrasonic transducer 16. The ultrasonic transmission waveguide 78 is isolated from the other elements of the elongated shaft assembly 14 by a protective sheath 80 and a plurality of isolation elements 82, such as silicone rings. The outer tubular sheath 56, the reciprocating tubular actuating member 58, and the ultrasonic transmission waveguide 78 are mechanically engaged by a pin 74. The switch assembly 28 comprises the toggle switch 30 and electrical elements 86a,b to electrically energize the ultrasonic transducer 16 in accordance with the activation of the first or second projecting knobs 30a, 30b.

In one example aspect, the outer tubular sheath 56 isolates the user or the patient from the ultrasonic vibrations of the ultrasonic transmission waveguide 78. The outer tubular sheath 56 generally includes a hub 76. The outer tubular sheath 56 is threaded onto the distal end of the handle assembly 12. The ultrasonic transmission waveguide 78 extends through the opening of the outer tubular sheath 56 and the isolation elements 82 isolate the ultrasonic transmission waveguide 24 from the outer tubular sheath 56. The outer tubular sheath 56 may be attached to the waveguide 78 with the pin 74. The hole to receive the pin 74 in the waveguide 78 may occur nominally at a displacement node. The waveguide 78 may screw or snap into the hand piece handle assembly 12 by a stud. Flat portions on the hub 76 may allow the assembly to be torqued to a required level. In one example aspect, the hub 76 portion of the outer tubular sheath 56 is preferably constructed from plastic and the tubular elongated portion of the outer tubular sheath 56 is fabricated from stainless steel. Alternatively, the ultrasonic transmission waveguide 78 may comprise polymeric material surrounding it to isolate it from outside contact.

In one example aspect, the distal end of the ultrasonic transmission waveguide 78 may be coupled to the proximal end of the ultrasonic blade 66 by an internal threaded connection, preferably at or near an antinode. It is contemplated that the ultrasonic blade 66 may be attached to the ultrasonic transmission waveguide 78 by any suitable means, such as a welded joint or the like. Although the ultrasonic blade 66 may be detachable from the ultrasonic transmission waveguide 78, it is also contemplated that the single element end effector (e.g., the ultrasonic blade 66) and the ultrasonic transmission waveguide 78 may be formed as a single unitary piece.

In one example aspect, the trigger 32 is coupled to a linkage mechanism to translate the rotational motion of the trigger 32 in directions 33a and 33b to the linear motion of the reciprocating tubular actuating member 58 in corresponding directions 60a and 60b (FIG. 2). The trigger 32 comprises a first set of flanges 98 with openings formed therein to receive a first yoke pin 94a. The first yoke pin 94a is also located through a set of openings formed at the distal end of the yoke 84. The trigger 32 also comprises a second set of flanges 96 to receive a first end of a link 92. A trigger pin 90 is received in openings formed in the link 92 and the second set of flanges 96. The trigger pin 90 is received in the openings formed in the link 92 and the second set of flanges 96 and is adapted to couple to the first and second portions 12a, 12b of the handle assembly 12 to form a trigger pivot point for the trigger 32. A second end of the link 92 is received in a slot formed in a proximal end of the yoke 84 and is retained therein by a second yoke pin 94b. As the trigger 32 is pivotally rotated about a pivot point formed by the trigger pin 90, the yoke translates horizontally along a longitudinal axis “T” in a direction indicated by arrows 60a,b.

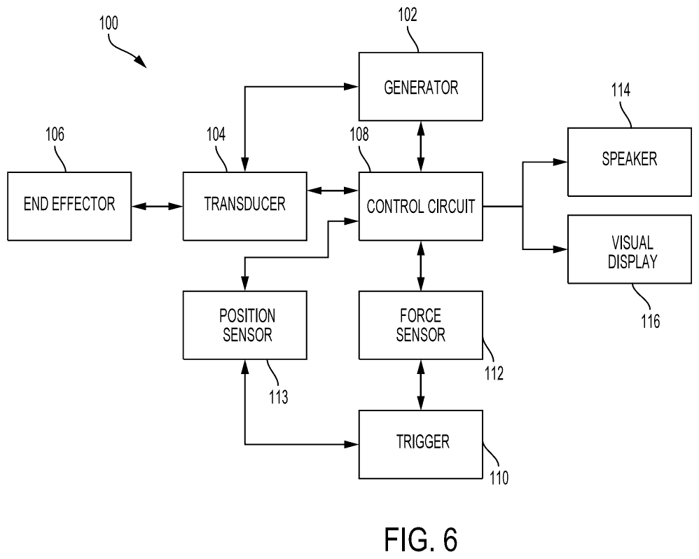

FIG. 6 illustrates a diagram of one aspect of a force feedback surgical device 100 which may include or implement many of the features described herein. For example, in one aspect, surgical device 100 may be similar to or representative of surgical instrument 10. The surgical device 100 may include a generator 102. The surgical device 100 may also include an ultrasonic end effector 106, which may be activated when a clinician operates a trigger 110. When the trigger 110 is actuated, a force sensor 112 may generate a signal indicating the amount of force being applied to the trigger 110. In addition to, or instead of force sensor 112, the device 100 may include a position sensor 113, which may generate a signal indicating the position of the trigger 110 (e.g., how far the trigger has been depressed or otherwise actuated). In one aspect, the position sensor 113 may be a sensor positioned with the outer tubular sheath 56 described above or reciprocating tubular actuating member 58 located within the outer tubular sheath 56 described above In one aspect, the sensor may be a Hall-effect sensor or any suitable transducer that varies its output voltage in response to a magnetic field. The Hall effect sensor may be used for proximity switching, positioning, speed detection, and current sensing applications. In one aspect, the Hall-effect sensor operates as an analog transducer, directly returning a voltage. With a known magnetic field, its distance from the Hall plate can be determined.

A control circuit 108 may receive the signals from the sensors 112 and/or 113. The control circuit 108 may include any suitable analog or digital circuit components. The control circuit 108 also may communicate with the generator 102 and/or the transducer 104 to modulate the power delivered to the end effector 106 and/or the generator level or ultrasonic blade amplitude of the end effector 106 based on the force applied to the trigger 110 and/or the position of the trigger 110 and/or the position of the outer tubular sheath 56 described above relative to the reciprocating tubular actuating member 58 located within the outer tubular sheath 56 described above (e.g., as measured by a Hall-effect sensor and magnet combination). For example, as more force is applied to the trigger 110, more power and/or a higher ultrasonic blade amplitude may be delivered to the end effector 106. According to various aspects, the force sensor 112 may be replaced by a multi-position switch.

According to various aspects, the end effector 106 may include a clamp or clamping mechanism, for example, such as that described above with respect to FIGS. 1-5. When the trigger 110 is initially actuated, the clamping mechanism may close, clamping tissue between a clamp arm and the end effector 106. As the force applied to the trigger increases (e.g., as sensed by force sensor 112) the control circuit 608 may increase the power delivered to the end effector 106 by the transducer 104 and/or the generator level or ultrasonic blade amplitude brought about in the end effector 106. In one aspect, trigger position, as sensed by position sensor 113 or clamp or clamp arm position, as sensed by position sensor 113 (e.g., with a Hall-effect sensor), may be used by the control circuit 108 to set the power and/or amplitude of the end effector 106. For example, as the trigger is moved further towards a fully actuated position, or the clamp or clamp arm moves further towards the ultrasonic blade (or end effector 106), the power and/or amplitude of the end effector 106 may be increased.

According to various aspects, the surgical device 100 also may include one or more feedback devices for indicating the amount of power delivered to the end effector 106. For example, a speaker 114 may emit a signal indicative of the end effector power. According to various aspects, the speaker 114 may emit a series of pulse sounds, where the frequency of the sounds indicates power. In addition to, or instead of the speaker 114, the device may include a visual display 116. The visual display 116 may indicate end effector power according to any suitable method. For example, the visual display 116 may include a series of light emitting diodes (LEDs), where end effector power is indicated by the number of illuminated LEDs. The speaker 114 and/or visual display 116 may be driven by the control circuit 108. According to various aspects, the device 100 may include a ratcheting device (not shown) connected to the trigger 110. The ratcheting device may generate an audible sound as more force is applied to the trigger 110, providing an indirect indication of end effector power. The device 100 may include other features that may enhance safety. For example, the control circuit 108 may be configured to prevent power from being delivered to the end effector 106 in excess of a predetermined threshold. Also, the control circuit 108 may implement a delay between the time when a change in end effector power is indicated (e.g., by speaker 114 or display 116), and the time when the change in end effector power is delivered. In this way, a clinician may have ample warning that the level of ultrasonic power that is to be delivered to the end effector 106 is about to change.

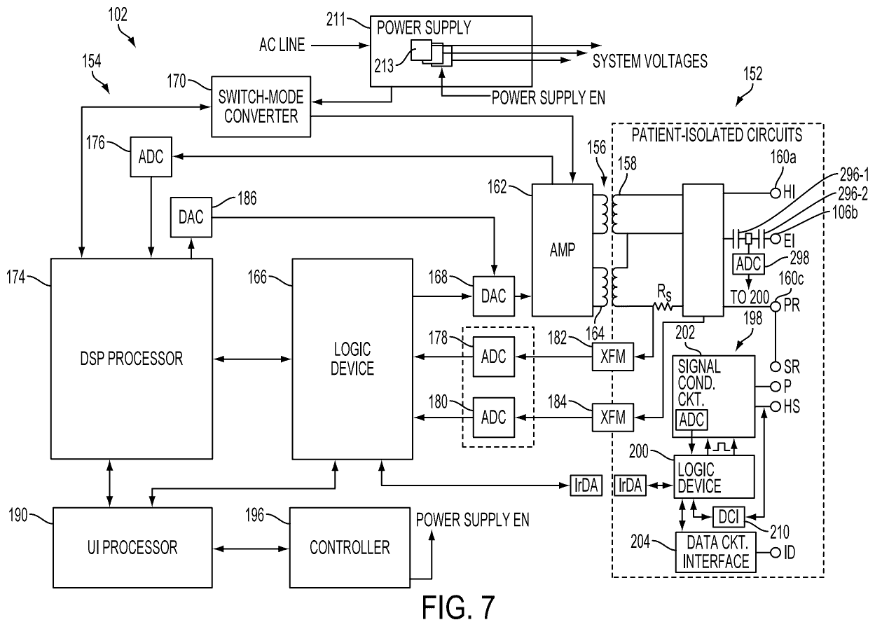

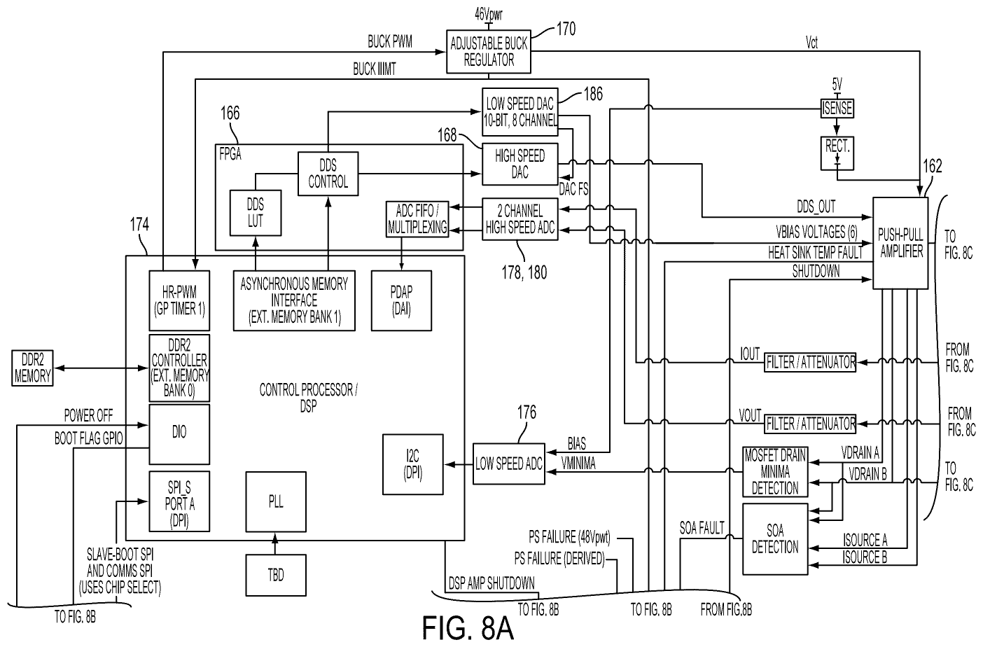

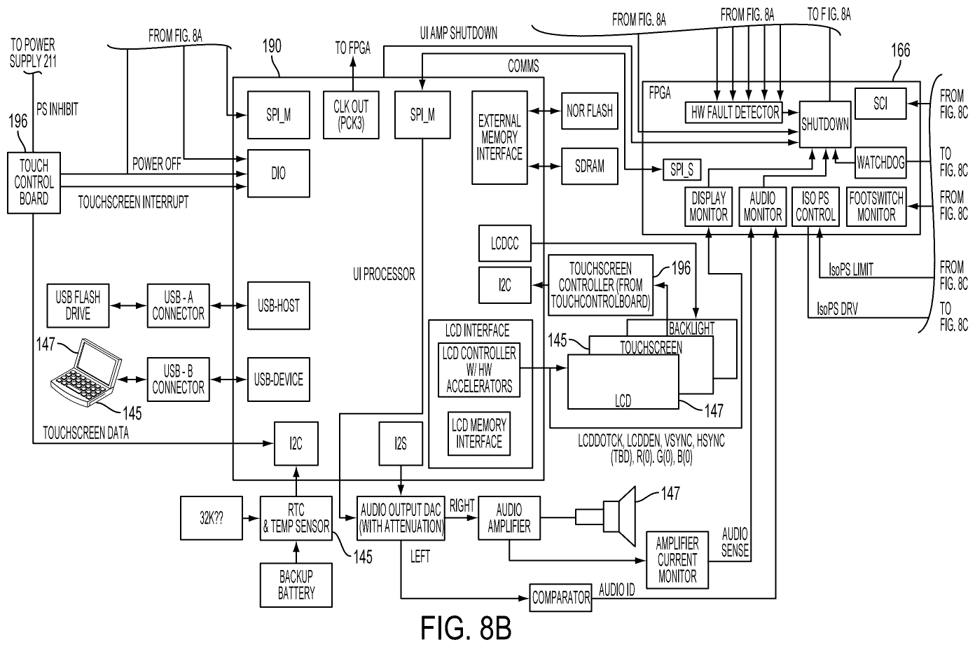

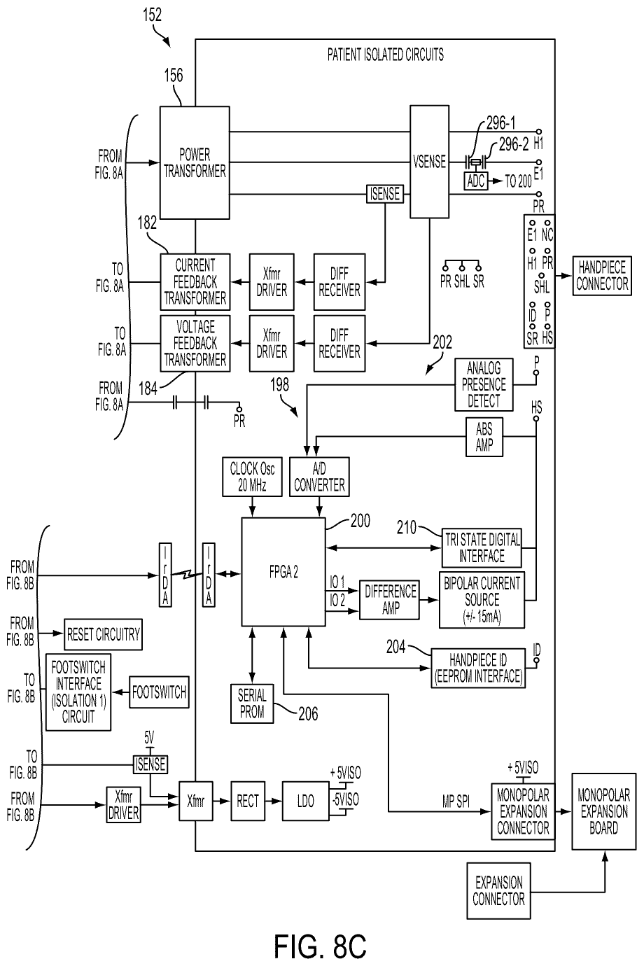

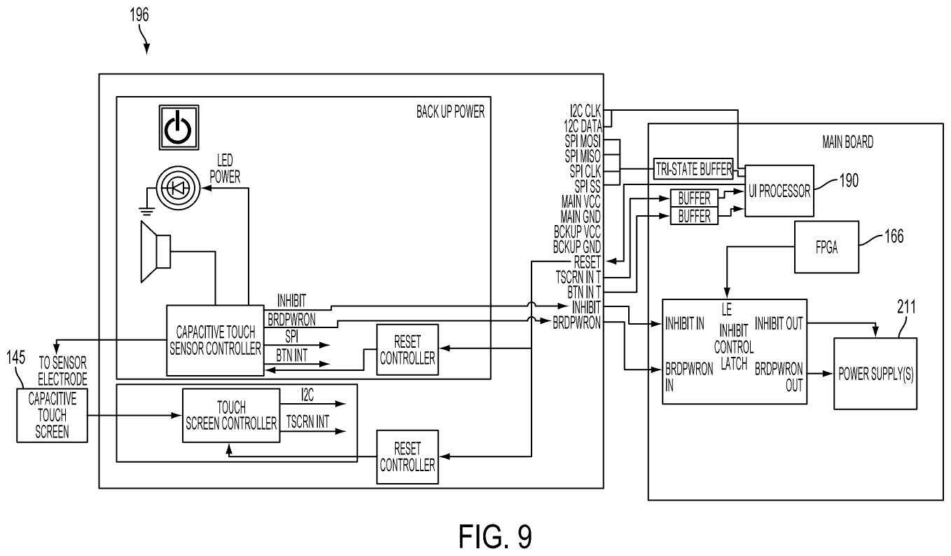

FIG. 7 is a simplified diagram of one aspect of the generator 102 which may provide for inductorless tuning, among other benefits. FIGS. 8A-8C illustrate an architecture of the generator 102 of FIG. 7 according to one aspect of the present disclosure. FIG. 9 illustrates a controller 196 for monitoring input devices and controlling output devices in accordance with one aspect of the present disclosure. With reference now to FIGS. 7-9, the generator 102 may comprise a patient isolated stage 152 in communication with a non-isolated stage 154 via a power transformer 156. A secondary winding 158 of the power transformer 156 is contained in the isolated stage 152 and may comprise a tapped configuration (e.g., a center-tapped or non-center tapped configuration) to define drive signal outputs 160a, 160b, 160c for outputting drive signals to different surgical devices, such as, for example, a surgical device 100, ultrasonic surgical instrument 10, or an electrosurgical device. In particular, drive signal outputs 160a, 160c may output a drive signal (e.g., a 420V RMS drive signal) to an ultrasonic instrument 10, and drive signal outputs 160b, 160c may output a drive signal (e.g., a 100V RMS drive signal) to an electro surgical device, with output 160b corresponding to the center tap of the power transformer 156. The non-isolated stage 154 may comprise a power amplifier 162 having an output connected to a primary winding 164 of the power transformer 156. In certain aspects the power amplifier 162 may comprise a push-pull amplifier, for example. The non-isolated stage 154 may further comprise a programmable logic device 166 for supplying a digital output to a digital-to-analog converter (DAC) 168, which in turn supplies a corresponding analog signal to an input of the power amplifier 162. In certain aspects the programmable logic device 166 may comprise a field-programmable gate array (FPGA), for example. The programmable logic device 166, by virtue of controlling the power amplifier's 162 input via the DAC 168, may therefore control any of a number of parameters (e.g., frequency, waveform shape, waveform amplitude) of drive signals appearing at the drive signal outputs 160a, 160b, 160c. In certain aspects and as discussed below, the programmable logic device 166, in conjunction with a processor (e.g., processor 17 4 discussed below), may implement a number of digital signal processing (DSP)-based and/or other control algorithms to control parameters of the drive signals output by the generator 102.

Power may be supplied to a power rail of the power amplifier 162 by a switch-mode regulator 170. In certain aspects the switch-mode regulator 170 may comprise an adjustable buck regulator, for example. The non-isolated stage 154 may further comprise a processor 174, which in one aspect may comprise a DSP processor such as an Analog Devices ADSP-21469 SHARC DSP, available from Analog Devices, Norwood, Mass., for example. In certain aspects the processor 174 may control operation of the switch-mode power converter 170 responsive to voltage feedback data received from the power amplifier 162 by the processor 174 via an analog-to-digital converter (ADC) 176. In one aspect, for example, the processor 174 may receive as input, via the ADC 176, the waveform envelope of a signal (e.g., an RF signal) being amplified by the power amplifier 162. The processor 174 may then control the switch-mode regulator 170 (e.g., via a pulse-width modulated (PWM) output) such that the rail voltage supplied to the power amplifier 162 tracks the waveform envelope of the amplified signal. By dynamically modulating the rail voltage of the power amplifier 162 based on the waveform envelope, the efficiency of the power amplifier 162 may be significantly improved relative to a fixed rail voltage amplifier schemes.

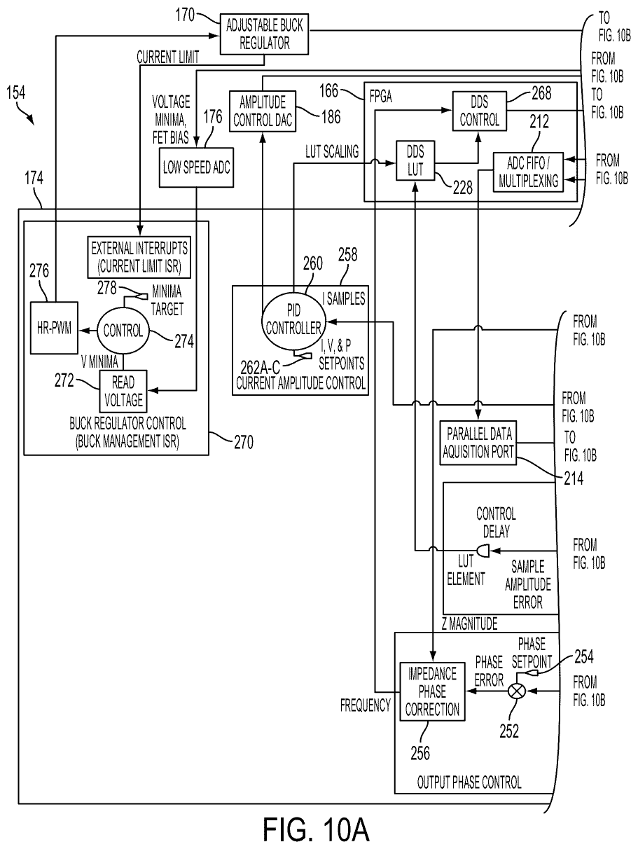

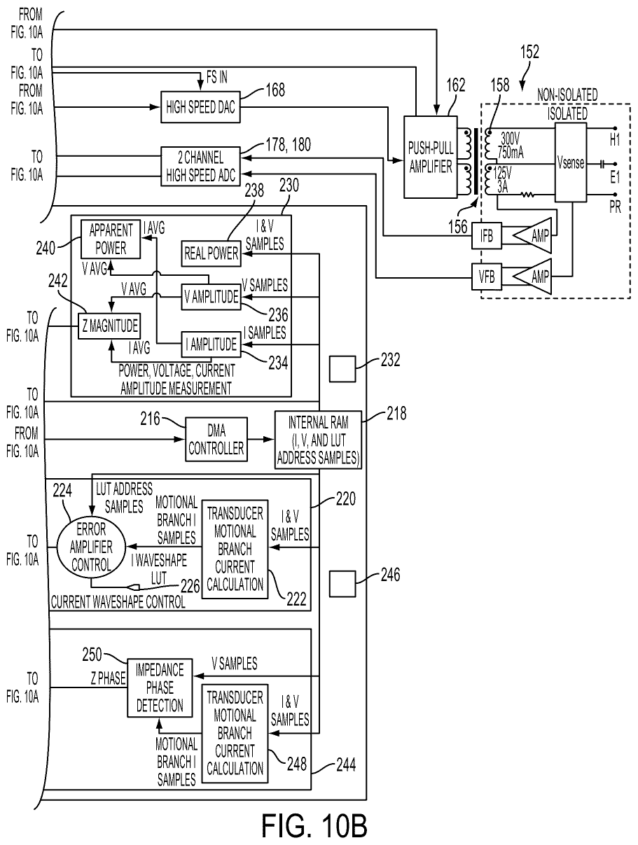

In certain aspects and as discussed in further detail in connection with FIGS. 10A and 10B, the programmable logic device 166, in conjunction with the processor 174, may implement a direct digital synthesizer (DDS) control scheme to control the waveform shape, frequency and/or amplitude of drive signals output by the generator 102. In one aspect, for example, the programmable logic device 166 may implement a DDS control algorithm 268 by recalling waveform samples stored in a dynamically-updated lookup table (LUT), such as a RAM LUT which may be embedded in an FPGA. This control algorithm is particularly useful for ultrasonic applications in which an ultrasonic transducer may be driven by a clean sinusoidal current at its resonant frequency. Because other frequencies may excite parasitic resonances, minimizing or reducing the total distortion of the motional branch current may correspondingly minimize or reduce undesirable resonance effects. Because the waveform shape of a drive signal output by the generator 102 is impacted by various sources of distortion present in the output drive circuit (e.g., the power transformer 156, the power amplifier 162), voltage and current feedback data based on the drive signal may be input into an algorithm, such as an error control algorithm implemented by the processor 174, which compensates for distortion by suitably pre-distorting or modifying the waveform samples stored in the LUT on a dynamic, ongoing basis (e.g., in real-time). In one aspect, the amount or degree of pre-distortion applied to the LUT samples may be based on the error between a computed motional branch current and a desired current waveform shape, with the error being determined on a sample-by sample basis. In this way, the predistorted LUT samples, when processed through the drive circuit, may result in a motional branch drive signal having the desired waveform shape (e.g., sinusoidal) for optimally driving the ultrasonic transducer. In such aspects, the LUT waveform samples will therefore not represent the desired waveform shape of the drive signal, but rather the waveform shape that is required to ultimately produce the desired waveform shape of the motional branch drive signal when distortion effects are taken into account.

The non-isolated stage 154 may further comprise an ADC 178 and an ADC 180 coupled to the output of the power transformer 156 via respective isolation transformers 182, 184 for respectively sampling the voltage and current of drive signals output by the generator 102. In certain aspects, the ADCs 178, 180 may be configured to sample at high speeds (e.g., 80 Msps) to enable oversampling of the drive signals. In one aspect, for example, the sampling speed of the ADCs 178, 180 may enable approximately 200× (depending on drive frequency) oversampling of the drive signals. In certain aspects, the sampling operations of the ADCs 178, 180 may be performed by a singleADC receiving input voltage and current signals via a two-way multiplexer. The use of high-speed sampling in aspects of the generator 102 may enable, among other things, calculation of the complex current flowing through the motional branch (which may be used in certain aspects to implement DDSbased waveform shape control described above), accurate digital filtering of the sampled signals, and calculation of real power consumption with a high degree of precision. Voltage and current feedback data output by the ADCs 178, 180 may be received and processed (e.g., FIFO buffering, multiplexing) by the programmable logic device 166 and stored in data memory for subsequent retrieval by, for example, the processor 174. As noted above, voltage and current feedback data may be used as input to an algorithm for pre-distorting or modifying LUT waveform samples on a dynamic and ongoing basis. In certain aspects, this may require each stored voltage and current feedback data pair to be indexed based on, or otherwise associated with, a corresponding LUT sample that was output by the programmable logic device 166 when the voltage and current feedback data pair was acquired. Synchronization of the LUT samples and the voltage and current feedback data in this manner contributes to the correct timing and stability of the pre-distortion algorithm.

In certain aspects, the voltage and current feedback data may be used to control the frequency and/or amplitude (e.g., current amplitude) of the drive signals. In one aspect, for example, voltage and current feedback data may be used to determine impedance phase. The frequency of the drive signal may then be controlled to minimize or reduce the difference between the determined impedance phase and an impedance phase setpoint (e.g., 0°), thereby minimizing or reducing the effects of ultrasonic distortion and correspondingly enhancing impedance phase measurement accuracy. The determination of phase impedance and a frequency control signal may be implemented in the processor 174, for example, with the frequency control signal being supplied as input to a DDS control algorithm implemented by the programmable logic device 166.

In another aspect, for example, the current feedback data may be monitored in order to maintain the current amplitude of the drive signal at a current amplitude setpoint. The current amplitude setpoint may be specified directly or determined indirectly based on specified voltage amplitude and power setpoints. In certain aspects, control of the current amplitude may be implemented by control algorithm, such as, for example, a proportional-integral-derivative (PID) or a proportional-integral (PI) control algorithm, in the processor 174. Variables controlled by the control algorithm to suitably control the current amplitude of the drive signal may include, for example, the scaling of the LUT waveform samples stored in the programmable logic device 166 and/or the full-scale output voltage of the DAC 168 (which supplies the input to the power amplifier 162) via a DAC 186.

The non-isolated stage 154 may further comprise a processor 190 for providing, among other things user interface (UI) functionality. In one aspect, the processor 190 may comprise an Atmel AT91 SAM9263 processor having an ARM 926EJ-S core, available from Atmel Corporation, San Jose, Calif., for example. Examples of UI functionality supported by the processor 190 may include audible and visual user feedback, communication with peripheral devices (e.g., via a Universal Serial Bus (USB) interface), communication with the footswitch 120, communication with an input device 145 (e.g., a touch screen display) and communication with an output device 146 (e.g., a speaker). The processor 190 may communicate with the processor 174 and the programmable logic device (e.g., via serial peripheral interface (SPI) buses). Although the processor 190 may primarily support UI functionality, it may also coordinate with the processor 174 to implement hazard mitigation in certain aspects. For example, the processor 190 may be programmed to monitor various aspects of user input and/or other inputs (e.g., touch screen inputs, footswitch 120 inputs, temperature sensor inputs) and may disable the drive output of the generator 102 when an erroneous condition is detected.

In certain aspects, both the processor 174 and the processor 190 may determine and monitor the operating state of the generator 102. For the processor 174, the operating state of the generator 102 may dictate, for example, which control and/or diagnostic processes are implemented by the processor 174. For the processor 190, the operating state of the generator 102 may dictate, for example, which elements of a user interface (e.g., display screens, sounds) are presented to a user. The processors 174, 190 may independently maintain the current operating state of the generator 102 and recognize and evaluate possible transitions out of the current operating state. The processor 174 may function as the master in this relationship and determine when transitions between operating states are to occur. The processor 190 may be aware of valid transitions between operating states and may confirm if a particular transition is appropriate. For example, when the processor 174 instructs the processor 190 to transition to a specific state, the processor 190 may verify that requested transition is valid. In the event that a requested transition between states is determined to be invalid by the processor 190, the processor 190 may cause the generator 102 to enter a failure mode.

The non-isolated stage 154 may further comprise a controller 196 for monitoring input devices 145 (e.g., a capacitive touch sensor used for turning the generator 102 on and off, a capacitive touch screen). In certain aspects, the controller 196 may comprise at least one processor and/or other controller device in communication with the processor 190. In one aspect, for example, the controller 196 may comprise a processor (e.g., a Mega168 8-bit controller available from Atmel) configured to monitor user input provided via one or more capacitive touch sensors. In one aspect, the controller 196 may comprise a touch screen controller (e.g., a QT5480 touch screen controller available from Atmel) to control and manage the acquisition of touch data from a capacitive touch screen.

In certain aspects, when the generator 102 is in a “power off” state, the controller 196 may continue to receive operating power (e.g., via a line from a power supply of the generator 102, such as the power supply 211 discussed below). In this way, the controller 196 may continue to monitor an input device 145 (e.g., a capacitive touch sensor located on a front panel of the generator 102) for turning the generator 102 on and off. When the generator 102 is in the power off state, the controller 196 may wake the power supply (e.g., enable operation of one or more DC/DC voltage converters 213 of the power supply 211) if activation of the “on/off” input device 145 by a user is detected. The controller 196 may therefore initiate a sequence for transitioning the generator 102 to a “power on” state. Conversely, the controller 196 may initiate a sequence for transitioning the generator 102 to the power off state if activation of the “on/off” input device 145 is detected when the generator 102 is in the power on state. In certain aspects, for example, the controller 196 may report activation of the “on/off” input device 145 to the processor 190, which in turn implements the necessary process sequence for transitioning the generator 102 to the power off state. In such aspects, the controller 196 may have no independent ability for causing the removal of power from the generator 102 after its power on state has been established.

In certain aspects, the controller 196 may cause the generator 102 to provide audible or other sensory feedback for alerting the user that a power on or power off sequence has been initiated. Such an alert may be provided at the beginning of a power on or power off sequence and prior to the commencement of other processes associated with the sequence.

In certain aspects, the isolated stage 152 may comprise an instrument interface circuit 198 to, for example, provide a communication interface between a control circuit of a surgical device (e.g., a control circuit comprising handpiece switches) and components of the non-isolated stage 154, such as, for example, the programmable logic device 166, the processor 174 and/or the processor 190. The instrument interface circuit 198 may exchange information with components of the non-isolated stage 154 via a communication link that maintains a suitable degree of electrical isolation between the stages 152, 154, such as, for example, an infrared (IR)-based communication link. Power may be supplied to the instrument interface circuit 198 using, for example, a low-dropout voltage regulator powered by an isolation transformer driven from the non-isolated stage 154.

In one aspect, the instrument interface circuit 198 may comprise a programmable logic device 200 (e.g., an FPGA) in communication with a signal conditioning circuit 202. The signal conditioning circuit 202 may be configured to receive a periodic signal from the programmable logic device 200 (e.g., a 2 kHz square wave) to generate a bipolar interrogation signal having an identical frequency. The interrogation signal may be generated, for example, using a bipolar current source fed by a differential amplifier. The interrogation signal may be communicated to a surgical device control circuit (e.g., by using a conductive pair in a cable that connects the generator 102 to the surgical device) and monitored to determine a state or configuration of the control circuit. The control circuit may comprise a number of switches, resistors and/or diodes to modify one or more characteristics (e.g., amplitude, rectification) of the interrogation signal such that a state or configuration of the control circuit is uniquely discernable based on the one or more characteristics. In one aspect, for example, the signal conditioning circuit 202 may comprise an ADC for generating samples of a voltage signal appearing across inputs of the control circuit resulting from passage of interrogation signal therethrough. The programmable logic device 200 (or a component of the nonisolated stage 154) may then determine the state or configuration of the control circuit based on the ADC samples.

In one aspect, the instrument interface circuit 198 may comprise a first data circuit interface 204 to enable information exchange between the programmable logic device 200 (or other element of the instrument interface circuit 198) and a first data circuit disposed in or otherwise associated with a surgical device. In certain aspects, a first data circuit 206 may be disposed in a cable integrally attached to a surgical device handpiece, or in an adaptor for interfacing a specific surgical device type or model with the generator 102. In certain aspects, the first data circuit may comprise a non-volatile storage device, such as an electrically erasable programmable read-only memory (EEPROM) device. In certain aspects and referring again to FIG. 7, the first data circuit interface 204 may be implemented separately from the programmable logic device 200 and comprise suitable circuitry (e.g., discrete logic devices, a processor) to enable communication between the programmable logic device 200 and the first data circuit. In other aspects, the first data circuit interface 204 may be integral with the programmable logic device 200.

In certain aspects, the first data circuit 206 may store information pertaining to the particular surgical device with which it is associated. Such information may include, for example, a model number, a serial number, a number of operations in which the surgical device has been used, and/or any other type of information. This information may be read by the instrument interface circuit 198 (e.g., by the programmable logic device 200), transferred to a component of the non-isolated stage 154 (e.g., to programmable logic device 166, processor 174 and/or processor 190) for presentation to a user via an output device 146 and/or for controlling a function or operation of the generator 102. Additionally, any type of information may be communicated to first data circuit 206 for storage therein via the first data circuit interface 204 (e.g., using the programmable logic device 200). Such information may comprise, for example, an updated number of operations in which the surgical device has been used and/or dates and/or times of its usage.

A surgical instrument may be detachable from a handpiece to promote instrument interchangeability and/or disposability. In such cases, known generators may be limited in their ability to recognize particular instrument configurations being used and to optimize control and diagnostic processes accordingly. The addition of readable data circuits to surgical device instruments to address this issue is problematic from a compatibility standpoint, however. For example, designing a surgical device to remain backwardly compatible with generators that lack the requisite data reading functionality may be impractical due to, for example, differing signal schemes, design complexity and cost. Aspects of instruments may use data circuits that may be implemented in existing surgical instruments economically and with minimal design changes to preserve compatibility of the surgical devices with current generator platforms.

Additionally, aspects of the generator 102 may enable communication with instrument-based data circuits. For example, the generator 102 may be configured to communicate with a second data circuit contained in an instrument of a surgical device. The instrument interface circuit 198 may comprise a second data circuit interface 210 to enable this communication. In one aspect, the second data circuit interface 210 may comprise a tri-state digital interface, although other interfaces may also be used. In certain aspects, the second data circuit may generally be any circuit for transmitting and/or receiving data. In one aspect, for example, the second data circuit may store information pertaining to the particular surgical instrument with which it is associated. Such information may include, for example, a model number, a serial number, a number of operations in which the surgical instrument has been used, and/or any other type of information. Additionally or alternatively, any type of information may be communicated to second data circuit for storage therein via the second data circuit interface 210 (e.g., using the programmable logic device 200). Such information may comprise, for example, an updated number of operations in which the instrument has been used and/or dates and/or times of its usage. In certain aspects, the second data circuit may transmit data acquired by one or more sensors (e.g., an instrument-based temperature sensor). In certain aspects, the second data circuit may receive data from the generator 102 and provide an indication to a user (e.g., an LED indication or other visible indication) based on the received data.

In certain aspects, the second data circuit and the second data circuit interface 210 may be configured such that communication between the programmable logic device 200 and the second data circuit can be effected without the need to provide additional conductors for this purpose (e.g., dedicated conductors of a cable connecting a handpiece to the generator 102). In one aspect, for example, information may be communicated to and from the second data circuit using a 1-wire bus communication scheme implemented on existing cabling, such as one of the conductors used transmit interrogation signals from the signal conditioning circuit 202 to a control circuit in a handpiece. In this way, design changes or modifications to the surgical device that might otherwise be necessary are minimized or reduced. Moreover, because different types of communications can be implemented over a common physical channel (either with or without frequency-band separation), the presence of a second data circuit may be “invisible” to generators that do not have the requisite data reading functionality, thus enabling backward compatibility of the surgical device instrument. In certain aspects, the isolated stage 152 may comprise at least one blocking capacitor 296-1 connected to the drive signal output 160b to prevent passage of DC current to a patient. A single blocking capacitor may be required to comply with medical regulations or standards, for example. While failure in single-capacitor designs is relatively uncommon, such failure may nonetheless have negative consequences. In one aspect, a second blocking capacitor 296-2 may be provided in series with the blocking capacitor 296-1, with current leakage from a point between the blocking capacitors 296-1, 296-2 being monitored by, for example, an ADC 298 for sampling a voltage induced by leakage current. The samples may be received by the programmable logic device 200, for example. Based on changes in the leakage current (as indicated by the voltage samples in the aspect of FIG. 7), the generator 102 may determine when at least one of the blocking capacitors 296-1, 296-2 has failed. Accordingly, the aspect of FIG. 7 may provide a benefit over single-capacitor designs having a single point of failure.

In certain aspects, the non-isolated stage 154 may comprise a power supply 211 for outputting DC power at a suitable voltage and current. The power supply may comprise, for example, a 400 W power supply for outputting a 48 VDC system voltage. The power supply 211 may further comprise one or more DC/DC voltage converters 213 for receiving the output of the power supply to generate DC outputs at the voltages and currents required by the various components of the generator 102. As discussed above in connection with the controller 196, one or more of the DC/DC voltage converters 213 may receive an input from the controller 196 when activation of the “on/off” input device 145 by a user is detected by the controller 196 to enable operation of, or wake, the DC/DC voltage converters 213.

FIGS. 10A and 10B illustrate certain functional and structural aspects of one aspect of the generator 102. Feedback indicating current and voltage output from the secondary winding 158 of the power transformer 156 is received by the ADCs 178, 180, respectively. As shown, the ADCs 178, 180 may be implemented as a 2-channel ADC and may sample the feedback signals at a high speed (e.g., 80 Msps) to enable oversampling (e.g., approximately 200× oversampling) of the drive signals. The current and voltage feedback signals may be suitably conditioned in the analog domain (e.g., amplified, filtered) prior to processing by the ADCs 178, 180. Current and voltage feedback samples from the ADCs 178, 180 may be individually buffered and subsequently multiplexed or interleaved into a single data stream within block 212 of the programmable logic device 166. In the aspect of FIGS. 10A and 10B, the programmable logic device 166 comprises an FPGA.

The multiplexed current and voltage feedback samples may be received by a parallel data acquisition port (PDAP) implemented within block 214 of the processor 174. The PDAP may comprise a packing unit for implementing any of a number of methodologies for correlating the multiplexed feedback samples with a memory address. In one aspect, for example, feedback samples corresponding to a particular LUT sample output by the programmable logic device 166 may be stored at one or more memory addresses that are correlated or indexed with the LUT address of the LUT sample. In another aspect, feedback samples corresponding to a particular LUT sample output by the programmable logic device 166 may be stored, along with the LUT address of the LUT sample, at a common memory location. In any event, the feedback samples may be stored such that the address of an LUT sample from which a particular set of feedback samples originated may be subsequently ascertained. As discussed above, synchronization of the LUT sample addresses and the feedback samples in this way contributes to the correct timing and stability of the pre-distortion algorithm. A direct memory access (DMA) controller implemented at block 216 of the processor 174 may store the feedback samples (and any LUT sample address data, where applicable) at a designated memory location 218 of the processor 174 (e.g., internal RAM).

Block 220 of the processor 174 may implement a pre-distortion algorithm for pre-distorting or modifying the LUT samples stored in the programmable logic device 166 on a dynamic, ongoing basis. As discussed above, pre-distortion of the LUT samples may compensate for various sources of distortion present in the output drive circuit of the generator 102. The pre-distorted LUT samples, when processed through the drive circuit, will therefore result in a drive signal having the desired waveform shape (e.g., sinusoidal) for optimally driving the ultrasonic transducer.

At block 222 of the pre-distortion algorithm, the current through the motional branch of the ultrasonic transducer is determined. The motional branch current may be determined using Kirchoffs Current Law based on, for example, the current and voltage feedback samples stored at memory location 218, a value of the ultrasonic transducer static capacitance Co (measured or known a priori) and a known value of the drive frequency. A motional branch current sample for each set of stored current and voltage feedback samples associated with a LUT sample may be determined.

At block 224 of the pre-distortion algorithm, each motional branch current sample determined at block 222 is compared to a sample of a desired current waveform shape to determine a difference, or sample amplitude error, between the compared samples. For this determination, the sample of the desired current waveform shape may be supplied, for example, from a waveform shape LUT 226 containing amplitude samples for one cycle of a desired current waveform shape. The particular sample of the desired current waveform shape from the LUT 226 used for the comparison may be dictated by the LUT sample address associated with the motional branch current sample used in the comparison. Accordingly, the input of the motional branch current to block 224 may be synchronized with the input of its associated LUT sample address to block 224. The LUT samples stored in the programmable logic device 166 and the LUT samples stored in the waveform shape LUT 226 may therefore be equal in number. In certain aspects, the desired current waveform shape represented by the LUT samples stored in the waveform shape LUT 226 may be a fundamental sine wave. Other waveform shapes may be desirable. For example, it is contemplated that a fundamental sine wave for driving main longitudinal motion of an ultrasonic transducer superimposed with one or more other drive signals at other frequencies, such as a third order ultrasonic for driving at least two mechanical resonances for beneficial vibrations of transverse or other modes, could be used.

Each value of the sample amplitude error determined at block 224 may be transmitted to the LUT of the programmable logic device 166 (shown at block 228 in FIG. 10A) along with an indication of its associated LUT address. Based on the value of the sample amplitude error and its associated address (and, optionally, values of sample amplitude error for the same LUT address previously received), the LUT 228 (or other control block of the programmable logic device 166) may pre-distort or modify the value of the LUT sample stored at the LUT address such that the sample amplitude error is reduced or minimized. It will be appreciated that such pre-distortion or modification of each LUT sample in an iterative manner across the entire range of LUT addresses will cause the waveform shape of the generator's output current to match or conform to the desired current waveform shape represented by the samples of the waveform shape LUT 226.

Current and voltage amplitude measurements, power measurements and impedance measurements may be determined at block 230 of the processor 174 based on the current and voltage feedback samples stored at memory location 218. Prior to the determination of these quantities, the feedback samples may be suitably scaled and, in certain aspects, processed through a suitable filter 232 to remove noise resulting from, for example, the data acquisition process and induced ultrasonic components. The filtered voltage and current samples may therefore substantially represent the fundamental frequency of the generator's drive output signal. In certain aspects, the filter 232 may be a finite impulse response (FIR) filter applied in the frequency domain. Such aspects may use the fast Fourier transform (FFT) of the output drive signal current and voltage signals. In certain aspects, the resulting frequency spectrum may be used to provide additional generator functionality. In one aspect, for example, the ratio of the second and/or third order ultrasonic component relative to the fundamental frequency component may be used as a diagnostic indicator. At block 234, a root mean square (RMS) calculation may be applied to a sample size of the current feedback samples representing an integral number of cycles of the drive signal to generate a measurement Irms representing the drive signal output current.

At block 236, a root mean square (RMS) calculation may be applied to a sample size of the voltage feedback samples representing an integral number of cycles of the drive signal to determine a measurement Vrms representing the drive signal output voltage. At block 238, the current and voltage feedback samples may be multiplied point by point, and a mean calculation is applied to samples representing an integral number of cycles of the drive signal to determine a measurement Pr of the generator's real output power.

At block 240, measurement Pa of the generator's apparent output power may be determined as the product Vrms·Irms.

At block 242, measurement Zm of the load impedance magnitude may be determined as the quotient Vrms/Irms.

In certain aspects, the quantities Irms, Vrms, Pr, Pa, and Zm determined at blocks 234, 236, 238, 240 and 242 may be used by the generator 102 to implement any of number of control and/or diagnostic processes. In certain aspects, any of these quantities may be communicated to a user via, for example, an output device 146 integral with the generator 102 or an output device 146 connected to the generator 102 through a suitable communication interface (e.g., a USB interface). Various diagnostic processes may include, without limitation, handpiece integrity, instrument integrity, instrument attachment integrity, instrument overload, approaching instrument overload, frequency lock failure, over-voltage, over-current, over-power, voltage sense failure, current sense failure, audio indication failure, visual indication failure, short circuit, power delivery failure, blocking capacitor failure, for example.

Block 244 of the processor 174 may implement a phase control algorithm for determining and controlling the impedance phase of an electrical load (e.g., the ultrasonic transducer) driven by the generator 102. As discussed above, by controlling the frequency of the drive signal to minimize or reduce the difference between the determined impedance phase and an impedance phase setpoint (e.g., 0°), the effects of ultrasonic distortion may be minimized or reduced, and the accuracy of the phase measurement increased.

The phase control algorithm receives as input the current and voltage feedback samples stored in the memory location 218. Prior to their use in the phase control algorithm, the feedback samples may be suitably scaled and, in certain aspects, processed through a suitable filter 246 (which may be identical to filter 232) to remove noise resulting from the data acquisition process and induced ultrasonic components, for example. The filtered voltage and current samples may therefore substantially represent the fundamental frequency of the generator's drive output signal.

At block 248 of the phase control algorithm, the current through the motional branch of the ultrasonic transducer is determined. This determination may be identical to that described above in connection with block 222 of the pre-distortion algorithm. The output of block 248 may thus be, for each set of stored current and voltage feedback samples associated with a LUT sample, a motional branch current sample.

At block 250 of the phase control algorithm, impedance phase is determined based on the synchronized input of motional branch current samples determined at block 248 and corresponding voltage feedback samples. In certain aspects, the impedance phase is determined as the average of the impedance phase measured at the rising edge of the waveforms and the impedance phase measured at the falling edge of the waveforms.

At block 252 of the of the phase control algorithm, the value of the impedance phase determined at block 222 is compared to phase setpoint 254 to determine a difference, or phase error, between the compared values.

At block 256 of the phase control algorithm, based on a value of phase error determined at block 252 and the impedance magnitude determined at block 242, a frequency output for controlling the frequency of the drive signal is determined. The value of the frequency output may be continuously adjusted by the block 256 and transferred to a DDS control block 268 (discussed below) in order to maintain the impedance phase determined at block 250 at the phase setpoint (e.g., zero phase error). In certain aspects, the impedance phase may be regulated to a oo phase setpoint. In this way, any ultrasonic distortion will be centered about the crest of the voltage waveform, enhancing the accuracy of phase impedance determination.

Block 258 of the processor 174 may implement an algorithm for modulating the current amplitude of the drive signal in order to control the drive signal current, voltage and power in accordance with user specified setpoints, or in accordance with requirements specified by other processes or algorithms implemented by the generator 102. Control of these quantities may be realized, for example, by scaling the LUT samples in the LUT 228 and/or by adjusting the full-scale output voltage of the DAC 168 (which supplies the input to the power amplifier 162) via a DAC 186. Block 260 (which may be implemented as a PID controller in certain aspects) may receive as input current feedback samples (which may be suitably scaled and filtered) from the memory location 218. The current feedback samples may be compared to a “current demand” Id value dictated by the controlled variable (e.g., current, voltage or power) to determine if the drive signal is supplying the necessary current. In aspects in which drive signal current is the control variable, the current demand Id may be specified directly by a current setpoint 262A (Isp). For example, an RMS value of the current feedback data (determined as in block 234) may be compared to user-specified RMS current setpoint Isp to determine the appropriate controller action. If, for example, the current feedback data indicates an RMS value less than the current setpoint Isp, LUT scaling and/or the full-scale output voltage of the DAC 168 may be adjusted by the block 260 such that the drive signal current is increased. Conversely, block 260 may adjust LUT scaling and/or the full-scale output voltage of the DAC 168 to decrease the drive signal current when the current feedback data indicates an RMS value greater than the current setpoint Isp.

In aspects in which the drive signal voltage is the control variable, the current demand Id may be specified indirectly, for example, based on the current required to maintain a desired voltage setpoint 262B (Vsp) given the load impedance magnitude Zm measured at block 242 (e.g. Id=Vsp/Zm). Similarly, in aspects in which drive signal power is the control variable, the current demand Id may be specified indirectly, for example, based on the current required to maintain a desired power setpoint 262C (Psp) given the voltage Vrms measured at blocks 236 (e.g. Id=Psp/Vrms).

Block 268 may implement a DDS control algorithm for controlling the drive signal by recalling LUT samples stored in the LUT 228. In certain aspects, the DDS control algorithm be a numerically-controlled oscillator (NCO) algorithm for generating samples of a waveform at a fixed clock rate using a point (memory location)-skipping technique. The NCO algorithm may implement a phase accumulator, or frequency-to-phase converter, that functions as an address pointer for recalling LUT samples from the LUT 228. In one aspect, the phase accumulator may be a D step size, modulo N phase accumulator, where D is a positive integer representing a frequency control value, and N is the number of LUT samples in the LUT 228. A frequency control value of D=1, for example, may cause the phase accumulator to sequentially point to every address of the LUT 228, resulting in a waveform output replicating the waveform stored in the LUT 228. When D>1, the phase accumulator may skip addresses in the LUT 228, resulting in a waveform output having a higher frequency. Accordingly, the frequency of the waveform generated by the DDS control algorithm may therefore be controlled by suitably varying the frequency control value. In certain aspects, the frequency control value may be determined based on the output of the phase control algorithm implemented at block 244. The output of block 268 may supply the input of (DAC) 168, which in turn supplies a corresponding analog signal to an input of the power amplifier 162.

Block 270 of the processor 174 may implement a switch-mode converter control algorithm for dynamically modulating the rail voltage of the power amplifier 162 based on the waveform envelope of the signal being amplified, thereby improving the efficiency of the power amplifier 162. In certain aspects, characteristics of the waveform envelope may be determined by monitoring one or more signals contained in the power amplifier 162. In one aspect, for example, characteristics of the waveform envelope may be determined by monitoring the minima of a drain voltage (e.g., a MOSFET drain voltage) that is modulated in accordance with the envelope of the amplified signal. A minima voltage signal may be generated, for example, by a voltage minima detector coupled to the drain voltage. The minima voltage signal may be sampled by ADC 176, with the output minima voltage samples being received at block 272 of the switch-mode converter control algorithm. Based on the values of the minima voltage samples, block 274 may control a PWM signal output by a PWM generator 276, which, in turn, controls the rail voltage supplied to the power amplifier 162 by the switch-mode regulator 170. In certain aspects, as long as the values of the minima voltage samples are less than a minima target 278 input into block 262, the rail voltage may be modulated in accordance with the waveform envelope as characterized by the minima voltage samples. When the minima voltage samples indicate low envelope power levels, for example, block 274 may cause a low rail voltage to be supplied to the power amplifier 162, with the full rail voltage being supplied only when the minima voltage samples indicate maximum envelope power levels. When the minima voltage samples fall below the minima target 278, block 274 may cause the rail voltage to be maintained at a minimum value suitable for ensuring proper operation of the power amplifier 162.

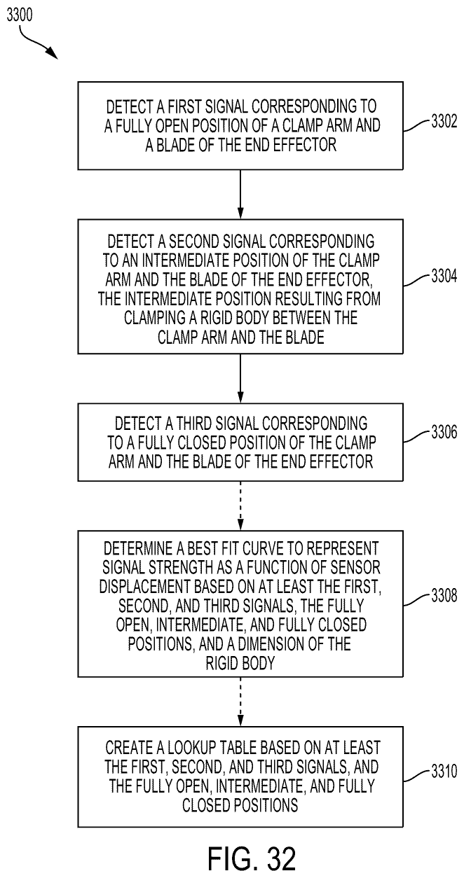

In one aspect, a method and/or apparatus may provide functionality for sensing a clamp arm position relative to a ultrasonic blade of an end effector, and a generator such as generator 102 and a controller such as control circuit 108 and/or controller 196 may be used to adjust a power output to the ultrasonic blade based on the clamp arm position. Referring now to FIG. 32, a process 3200 for controlling an end effector is shown. The process 3200 may be executed at least in part by a processor which may be in communication with or may be part of one or more of generator 102, control circuit 108, and/or controller 196. Referring now to FIG. 32, a process 3300 for calibrating a controller for an end effector is shown. The process 3200 may be executed at least in part by a processor which may be in communication with or may be part of one or more of generator 102, control circuit 108, and/or controller 196.

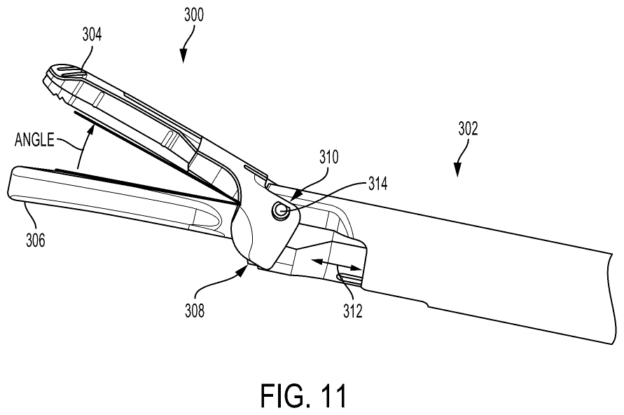

Referring now to FIG. 11, an example end effector 300 and shaft 302 are shown. The clamp arm 304 may have a position (e.g., represented by the “angle” arrow or a displacement) relative to the ultrasonic blade 306, which may be measured using one or more sensors such as Hall-effect sensor. Sensing the position of the clamp arm relative to the ultrasonic blade may provide relevant device information enabling new capabilities such as the ability to sense thickness, quantity, or types of tissues clamped inside the jaws. In one aspect, the process 3200 of FIG. 32 may determine 3220 a type of tissue between the clamp arm and the ultrasonic blade based on a signal (from, e.g., a Hall-effect sensor). Further, using a processor and/or memory, one or more algorithms (e.g., for sealing a vessel without transection) may be chosen based on the thickness, quantity, or type of tissue determined to be clamped inside the jaws.

Ultrasonic blade 306 may deliver a tissue effect through mechanical vibration to tissues and/or blood vessels. Clamp arm 304 may pivot about point 314, which may represent a connection between the clamp arm and an outer tube 310. An inner tube 308 may move back and forth and may drive closure of the clamp arm 304 on ultrasonic blade 306. In various aspects, it may be desirable to measure the angle between the clamp arm 304 and the ultrasonic blade 306.

In one aspect, the position of clamp arm 304 relative to ultrasonic blade 306 (e.g., during activation) may be approximated through a coupling with the inner tube 308. The inner tube 308 may be linked to the clamp arm 304 and may be similar to the reciprocating tubular actuating member 58 located within the outer tubular sheath 56. The outer tube 310, which may be similar to the outer tubular sheath 56, and/or ultrasonic blade 306, may be used to determine a position and/or angle of the clamp arm 304 relative to ultrasonic blade 306. The outer tube 310 may be static and in one aspect may be linked to clamp arm 304. As result, using the techniques and features described herein, the movement (e.g., represented with the bidirectional arrow 312) of the inner tube 308 relative to the outer tube 310 may be measured and used to approximate the claim arm position.

Referring briefly to FIG. 32, process 3200 may detect 3202 a signal (e.g., at a Hall-effect sensor) in response to movement of a first tube relative to a second tube, the first tube driving movement of a clamp arm of the end effector. The first tube may be, for example, similar to reciprocating tubular actuating member 58 and the second tube may be, for example, similar to outer tubular sheath 56. In other words, as described in FIG. 32, the first tube may be an inner tube and the second tube is an outer tube. The inner tube may be moveable 3208 relative to the outer tube. The outer tube may be static relative to the inner tube. The process 3200 may detect 3210 the signal using a Hall-effect sensor and a magnet positioned on the first tube.

Use of Hall-effect sensors will be described herein with respect to various aspects of the present disclosure, however other types of sensors may be used to measure the movement 312. For example, linear variable differential transformers (LVDT), rotary variable differential transformer, piezoelectric transducers, potentiometers, photo electric sensors may be used to measure the movement 312. Furthermore, Hall-effect sensors and suitable equivalents may be used to measure the position of two bodies relative to one another through the use of a small electronic board and magnets.

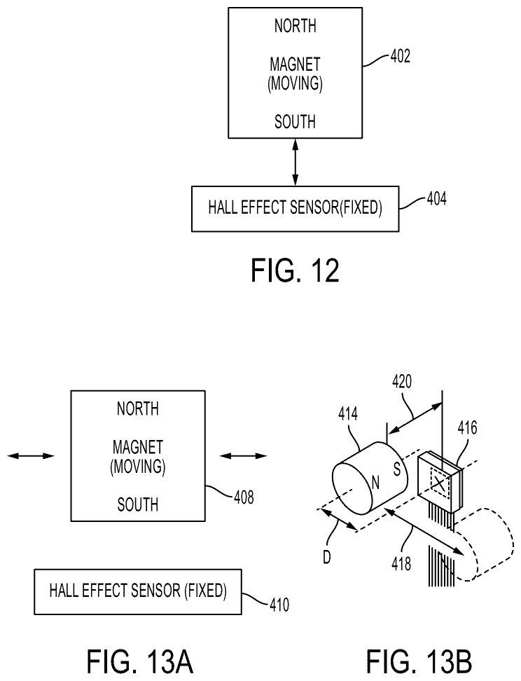

Referring now to FIG. 12, a representation of an example Hall-effect sensor is shown. A magnet 402 may have north and south poles which move in a line perpendicular to the face of the Hall sensor 404, which may be in a fixed position. Referring now to FIG. 13A, another representation of an example Hall-effect sensor is shown. A magnet 408 may have north and south poles moving in a line parallel to the face of the Hall-effect sensor 410, which may be in a fixed position. Referring now to FIG. 13B, another representation of an example Hall-effect sensor is shown. A magnet 414 may have north and south poles moving in a line (418) parallel to the face of the Hall-effect sensor 416, which may be in a fixed position. The magnet may have diameter D and the magnet and Hall-effect sensor 416 may have total effective air gap (TEAG) 420. This configuration may allow for a very sensitive measurement of movement over small distances with the appropriate magnet-sensor combination.

The Hall-effect sensor may include a small electronic chip which may sense magnetic fields and change its electrical output based on the relative proximity of the magnet or the strength of the magnetic fields to the Hall-effect sensor. As the magnet moves across the face of the Hall-effect sensor (e.g., marked “X”) and gets closer to being directly in front of the face, an output signal of the Hall-effect sensor may change and be used to determine a position of the magnet relative to the Hall-effect sensor. In one aspect, the magnet may not cause much of a change in the output signal of the Hall-effect sensor. For example, using a magnet and Hall-effect sensor having particular characteristics, the magnet being more than 1.5 inches or further distances from the Hall-effect sensor may produce very little in terms of the output signal, but as the magnet moves closer and closer to the Hall-effect sensor, the electrical output changes more rapidly such that a very discernable signal change occurs in response to small motions of the magnet as it is moved closer to a critical position. The electrical response of the Hall-effect sensor at various positions of the magnet may be used to create a best fit curve. For example, the voltage output of the Hall-effect sensor as a function of the displacement of the magnet may be determined.

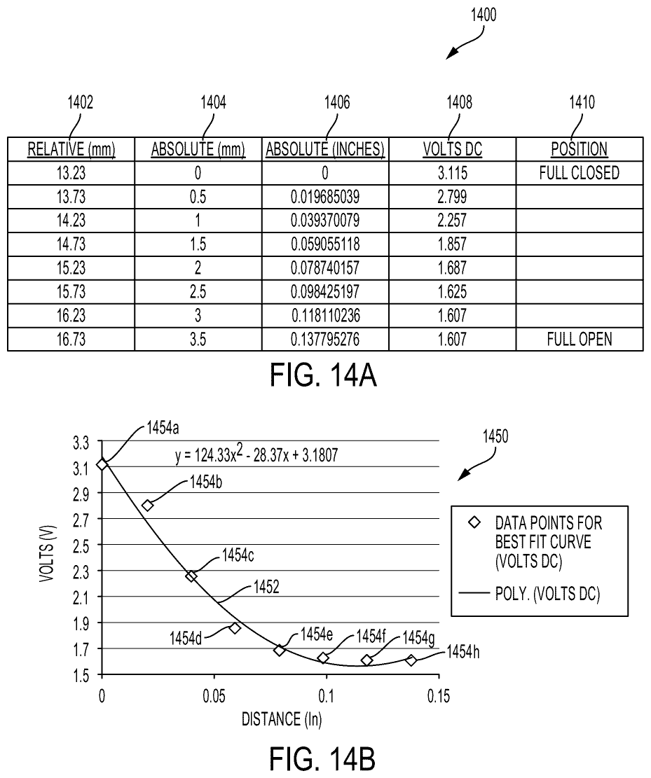

FIG. 14A is a table 1400 of output voltage of a Hall-effect sensor as a function of distance as a clamp arm moves from a fully closed position to a fully open position in accordance with the present disclosure. Relative distance (mm) is listed in the first column 1402. Absolute distance (mm) is listed in the second column 1404 and absolute distance in inches is listed in the third column 1406. Output voltage of the Hall-effect sensor is listed in the fourth column 1408 and clamp arm position is listed in the fifth column 1410, where the uppermost cell indicates the clamp arm in the fully closed position and the lowermost cell indicates the clamp arm in the fully open position.

Referring now to FIGS. 14A and 14B, a table 1400 and a graph 1450 of the output voltage of a Hall-effect sensor (y-axis) as a function of the displacement (x-axis) and related data are shown. In this example, the sensitivity of a prototyped Hall-effect sensor/magnet combination is shown as a relatively small linear movement (e.g., 0.100″) may result in a 1.5 volts signal change. This signal change may be read by a generator (e.g., generator 102) and used to make determinations about ultrasonic blade displacement, or provide auditory, tactile and/or other feedback to a user (e.g., via speaker 114 and/or visual display 116). A best fit curve 1452 may be determined from the plotted data points 145a-h (e.g., one or more of relative displacement, absolute displacement, voltage output, and position) and a polynomial equation for output voltage of the Hall-effect sensor (y-axis) as a function of the displacement (x-axis) of the magnet may result. The best fit curve may be of the 2nd, 3rd, 4th . . . nth order. The data points 1454a-h and/or the best fit curve 1452 may be used to create a lookup table stored in a memory and/or the resulting equation may be executed in a processor in order to determine, for example, a displacement for the magnet (and a corresponding clamp arm position) given a specific output voltage of the Hall-effect sensor. In this way, turning briefly to FIG. 32, the process 3200 may determine 3204 a clamp arm position of the end effector relative to a ultrasonic blade of the end effector based on the signal (from, e.g., Hall-effect sensor voltage output).

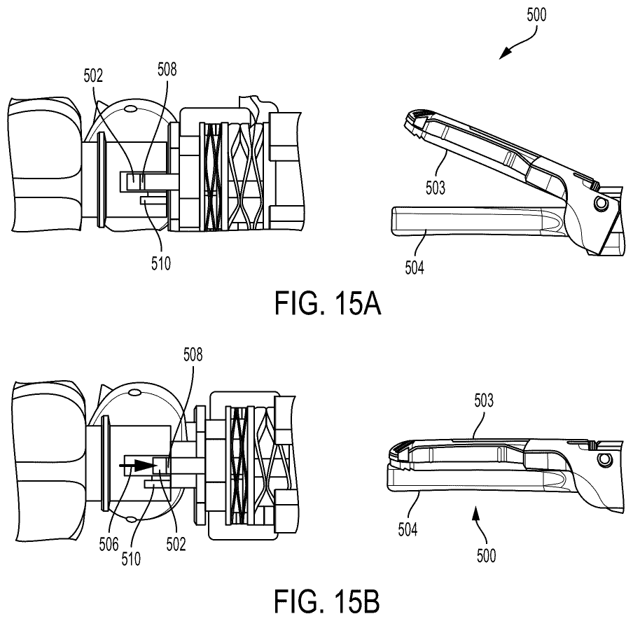

Turning now to FIG. 15A there is shown a top view of a Hall-effect sensor 510 and magnet 508 configurations in a surgical instrument and corresponding open jaws end effector 500 position in accordance with one aspect of the present disclosure and FIG. 15B is a top view of the Hall-effect sensor 510 and magnet 508 configurations in a surgical instrument and corresponding closed jaws end effector 500 position in accordance with one aspect of the present disclosure. In one aspect, as shown in FIGS. 15A and 15B, the voltage output of the Hall-effect sensor 50 is 1.6 VDC when the jaws of the end effector 500 are open and 3.1 VDC when the jaws of the end effector 500 are closed.

Referring now to FIGS. 15A and 15B, one aspect of a Hall-effect sensor 510 and magnet 508 combination are shown as implemented in a surgical device such as one or more of those discussed herein. FIGS. 15A and 15B show two images of top-down views of the example. An inner threaded collar 502 may be attached to a magnet 508. As a trigger of the surgical device is closed, a clamp arm 504 of the end effector 500 comes into closer contact with a ultrasonic blade 504, and the magnet 508 moves further proximal as shown in the top-down views. As the magnet 508 moves (in a direction indicated by arrow 506), the voltage potential of the Hall-effect sensor 510 changes. The magnet 508 positioned on the first tube relative to the Hall-effect sensor 510 may move as the first tube drives movement of the clamp arm 503 of the end effector 500.

It should be noted that while various aspects discussed herein are described to include an outer tube that is static and an inner tube that drives motion of the clamp arm, other configurations are possible and within the scope of the present disclosure. For example, in various aspects, an outer tube may drive the motion of the clamp arm and the inner tube may be static. Additionally, while various aspects discussed herein are described to include a Hall-effect sensor 510 and/or integrated circuit (e.g., chip) that is static and a magnet 508 that moves as the clamp arm 500 moves, other configurations are possible and within the scope of the present disclosure. For example, in various aspects, the Hall-effect sensor 510 may move as the clamp arm 503 moves and the magnet may be static. Many combinations are possible, including a fixed outer tube and a moveable inner, a moving magnet 508 and a stationery Hall-effect sensor 510 or other sensing circuit, a moving Hall-effect sensor 510 or other sensing circuit and a stationary magnet 508, a moveable outer tube and a fixed inner tube, a fixed magnet in one of the inner and outer tubes, and/or a moving magnet in one of the inner and outer tubes. The Hall-effect sensor 510 or other circuit may be mounted to the moving part (e.g., inner or outer tube) or mounted to the stationary part (e.g., inner or outer tube), as long as flexible electrical connections are considered and motion can be achieved.

As shown in FIG. 15A, the inner threaded collar 502 with attached magnet 508 is positioned further left than in FIG. 15B, and the corresponding end effector 500 has an open jaw, e.g., open clamp arm 503. As the user pulls the trigger and closes the end effector 500, multiple springs and the inner threaded collar 502 move (in the direction indicated by arrow 506) the clamp arm 503 is driven closed or is driven to grafting tissue captured in between the clamp arm 503 and the ultrasonic blade 504. A Hall-effect sensor 510 and a magnet 508 is shown, which may be cylindrical, moves over the Hall-effect sensor 510, as the clamp arm 503 closes towards the ultrasonic blade 504.

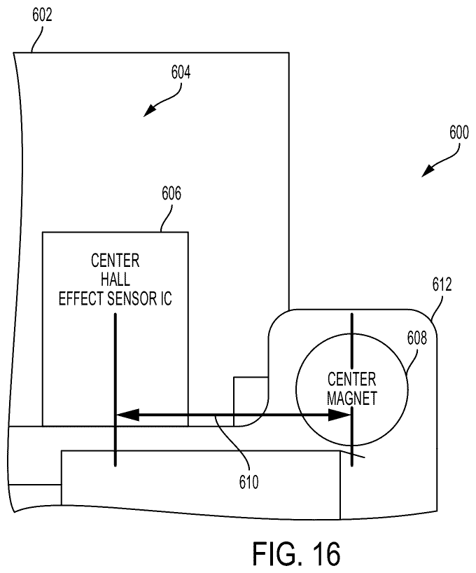

Referring now to FIG. 16, there is shown a plan view of a system 600 comprising a Hall-effect sensor 602 and magnet 606 arrangement. The Hall-effect sensor 602 includes a circuit board 604 and an integrated circuit 606. The magnet 608 moves back and forth along line 610 as the clamp arm is closed and opened. As the magnet 608 moves towards the center of the Hall effect integrated circuit 606 the sensitivity of the Hall-effect sensor 602 changes and the output signal increases. A carrier 612 for the magnet 608 may be coupled to the inner tube that drives the clamp arm. In one aspect, as the inner tube is pulled towards the handle of the surgical instrument (e.g., by the trigger), the jaw closes (e.g., clamp arm) closes. The magnet 608 is connected to an extended leg of the threaded inner collar of the outer tube.

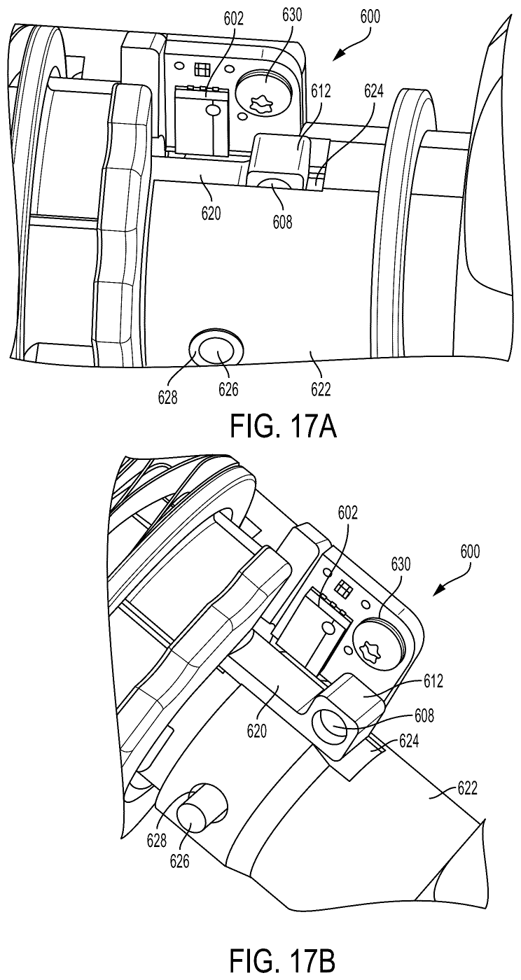

FIGS. 17A and 17B illustrates different views of the system 600 comprising a Hall-effect sensor 602 and magnet 608 configurations in the context of a surgical instrument in accordance with one aspect of the present disclosure. With reference to FIGS. 17A and 17B, the Hall-effect sensor 602 is shown positioned within a surgical instrument. The Hall-effect sensor 602 is positioned on the threaded inner collar 620 of the outer tube 622. A slot 624 is defined in a rotation knob of the outer tube 622 to allow the magnet 608 to travel. The magnet 608 is positioned within the carrier 612, which is slidably movable within the slot 624. For example, the Hall-effect sensor 602 as described herein may be static and is attached to a rotation knob such that it can rotate around the centerline of the ultrasonic blade. A pin 626 may be positioned within an aperture 628 through both the rotation knob and the Hall-effect sensor 602 and through a center ultrasonic blade portion. As a result, the ultrasonic blade does not move axially, but the inner tube is able to move axially right and left of the pin 626. A threaded connection 630 is made of nylon or any other suitable material with minimum magnetic flux.

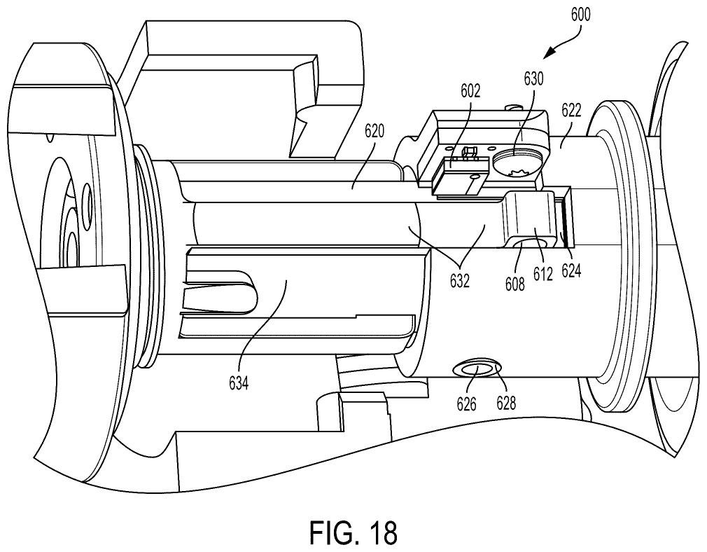

FIG. 18 illustrates a Hall-effect sensor 602 and magnet 608 configuration in the context of a surgical instrument in accordance with the present disclosure. Referring now to FIG. 18, a shaft of a surgical instrument is shown and the magnet 608 is positioned within the carrier 612. A magnet movement 632 is coupled to the inner tube 634. The magnet 608 may be coupled with snap fits to a threaded collar 638 of the inner tube 634. The Hall-effect sensor 602 as described herein is static and is attached to a rotation knob such that it can rotate around the centerline of the ultrasonic blade.

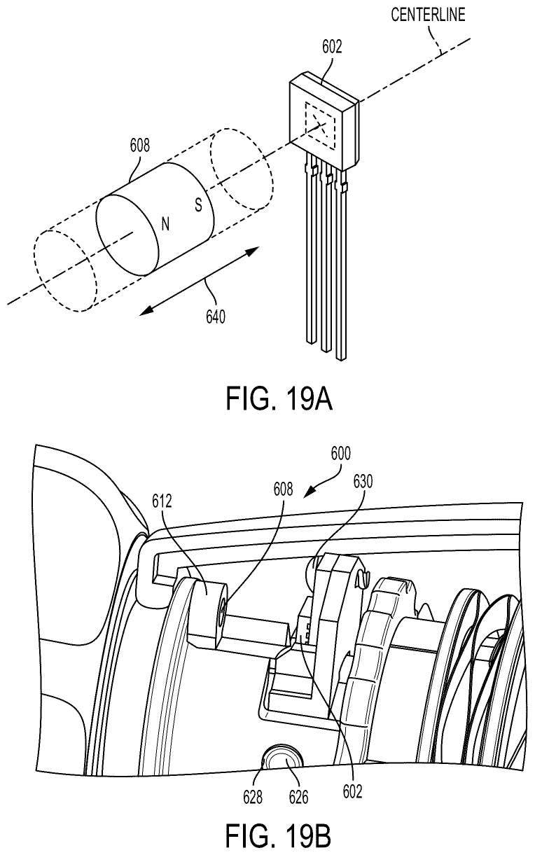

FIG. 19A illustrates a Hall-effect sensor 602 and magnet 608 configuration in accordance with one aspect of the present disclosure. FIG. 19B is a detailed view of the Hall-effect sensor 602 and magnet 608 configuration in the context of a surgical instrument in accordance with the present disclosure. Referring now to FIGS. 19A and 19B, in one aspect the Hall-effect sensor 602 and magnet 608 configuration is located on a shaft of a surgical instrument. In one aspect, the pole faces of the magnet 608 and the Hall-effect sensor 602 move in line with one another. In FIGS. 17A, 17B, 19A, and 19B, the Hall-effect sensor 602 is stationary while the magnet 608 moves in connection with the clamp arm. In one aspect, the inner threaded collar is configure to carry the magnet 608 and may be directly connected to the inner tube. In this way, the Hall-effect sensor 602 may be positioned in a different way on the rotation knob such that the faces of the magnet 608 and the Hall-effect sensor 602 come together in a perpendicular manner as shown by the motion arrow 640.

In one aspect, an ultrasonic algorithm or process may be used to enable a surgical device to seal tissue without transection. The implementation of this algorithm or process may require measuring clamp arm position relative to the ultrasonic blade of an end effector. A method can be used to sense the clamp arm position relative to the ultrasonic blade as described herein and that positioning can be consistently calibrated during manufacturing, as will be described below, such that estimates of thickness of tissue can be made. For example, an algorithm or process that is fed information about quantity of tissue can react as that quantity changes. This may allow the surgical device to treat the tissue without completely transecting a vessel.

Turning now briefly to FIG. 32, once the clamp arm position relative to ultrasonic blade is known, how the ultrasonic blade vibrates can be adjusted to get different tissue effects. In this way, process 3200 may adjust 3206 a power output to the ultrasonic blade of the end effector based on the clamp arm position. For example, process 3200 may adjust 3214 the power output to the ultrasonic blade of the end effector using an ultrasonic transducer based on a voltage change in a Hall-effect sensor.

Typically, end effectors may be used to coagulate and cuts vessels at the same time. However, using the techniques and features described herein, an end effector may be used to seal a carotid or vessel without actually transecting it, as may be desired by a surgeon. With information on the clamp arm position, a Travel Ratio (TR) can be calculated, whereby if the clamp arm is in the completely closed position with nothing captured in the end effector, the sensor (e.g., Hall-effect sensor) may indicate a TR of 1. For example, for illustrative purposes only, let XT represent a relative clamp arm position at any given time in activation, X1 be a claim arm position when the surgical device is fully clamped with no tissue, and X2 be a clamp arm position at a beginning of activation, with tissue grasped in the end effector, where:

$$TR = \frac{X 1 - XT}{X 1 - X 2}$$

Continuing with the example above, X1 may be a value programmed into the surgical device for the clamp arm position when the jaws are fully closed and nothing is captured in the end effector. X2 may be the clamp arm position at the start of an activation such that if a vessel is attached in the end effector and the clamp arm is closed all the way, the clamp arm may be squeezing the vessel down but with some distance to travel before the vessel is transected and the clamp arm is directly opposite the ultrasonic blade with full contact. XT may change dynamically as it is the clamp arm position at any given time.

For example, at the very beginning of activation TR may be zero, as X1 may be set to represent the clamp arm position being fully closed with nothing captured. X2, at the very beginning of the activation, when the clamp arm is touching a vessel, may provide a relative thickness before firing the ultrasonic blade. XT may be the value in the equation that is updating continuously with time as the clamp arm travels further and compresses and starts to cut the tissue. In one aspect, it may be desirable to deactivate (e.g., stop firing) the ultrasonic blade when the clamp arm has traveled 70% or 0.7. Thus, it may be empirically determined beforehand that a desired TR is 0.7 of the way between the clamp arm being closed with a full bite of tissue and being fully closed with nothing in between the clamp arm and ultrasonic blade.

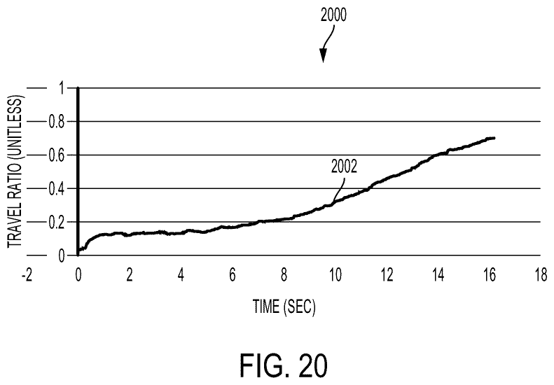

The TR of 0.7 has been described for illustrative purposes only and may depend on many parameters. For example, the desired TR for the point at which the ultrasonic blade will be shut-off may be based on vessel size. The TR may be any value observed to work for treating a given tissue or vessel without transection. Once the desired position is known, the vibrating of the ultrasonic blade may be adjusted based on the desired position. FIG. 20 is a graph 2000 of a curve 2002 depicting Travel Ratio (TR) along the y-axis, based on Hall-effect sensor output voltage, as a function of Time (Sec) along the x-axis. As shown in FIG. 20, the desired TR is 0.7, meaning that the ultrasonic blade is deactivated (e.g., stop firing) when the clamp arm has traveled 70% or 0.7. This is relative to a clamp arm on a vessel with a ultrasonic blade firing where the desired end TR was 0.7. In the particular example of FIG. 20, the ultrasonic blade was activated (e.g., firing) on a carotid and shut off at the TR of 0.7 after about 16 seconds.

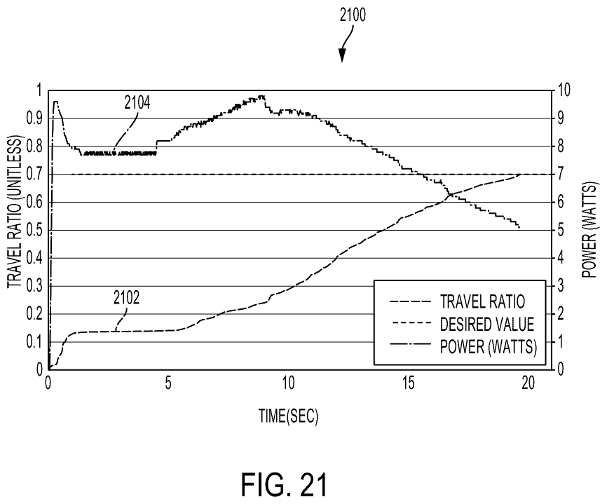

In one aspect, it may be desirable to use a proportional-integral controller. FIG. 21 is a graph 2100 of a first curve 2102 depicting Travel Ratio (TR) along the left y-axis, based on Hall-effect sensor output voltage, as a function of Time (Sec) along the x-axis. A second curve 2104 depicts Power (Watts) along the right y-axis as a function of Time (Sec) along the x-axis. The graph 2100 provides an example of what can be accomplished with a proportional-integral (PI) controller. The Travel Ratio (TR) curve 2102 is represented on the graph 2100 by the line marked “TRAVEL RATIO.” The goal or Desired Value for the Travel Ratio may be 0.7, as shown by the line marked “DESIRED VALUE,” although various other values may be used. The Power output curve 2104 represents power through the ultrasonic blade and is shown and marked “POWER (WATTS).”

Turning now briefly to FIG. 32, there is shown the process 3200 may adjust 3216 the power output to the ultrasonic blade of the end effector dynamically, based on the travel ratio that changes as the clamp arm approaches the ultrasonic blade. For example, as the clamp arm moves towards the ultrasonic blade and the Desired Value is approached, the amount of power output to the ultrasonic blade and into the tissue may be reduced. This is because the ultrasonic blade will cut the tissue with enough power. However, if the power being output is reduced over time as the Desired Value is approached (where a full transection may be represented by a Travel Ratio of 1), the chance that the tissue is transected may be drastically reduced. In this way, effective sealing may be achieved without cutting the tissue as may be desired by the surgeon.

Turning back to FIG. 21, there is shown the Power output curve 2104 shown in FIG. 21 may represent the power applied with a drive signal to a transducer stack to activate (e.g., fire) the ultrasonic blade. The Power value may be proportional to the movement of the clamp arm portion of the end effector and delivered to the tissue and the Power curve may represent voltage and current applied to the ultrasonic transducer. In one aspect, the ultrasonic generator (e.g., generator 102) may read the voltage output data from the Hall-effect sensor and, in response, send commands for how much voltage and current to provide to the transducer to drive the ultrasonic blade as desired. As the clamp arm portion of the end effector is moved and the desired value is approached, the ultrasonic blade may be forced to deliver less energy to the tissue and reduce the likelihood of cutting the tissue.

As the ultrasonic blade is powered, the ultrasonic blade will effect the tissue or the vessel such that friction at the interface of the ultrasonic blade and the tissue causes heat to drive the moisture from and dry out the tissue. During this process, the clamp arm portion is able to increasingly compress the tissue as the seal develops. As the TR increases over time the tissue flattens by applying more pressure with the clamp arm as the tissue dries out. In this way, a PI controller may be used to cook the tissue from a beginning point (where TR=0) to a certain second position by controlling power output to effectively seal large vessels. With the PI controller, as the TR approaches the Desired Value, the ultrasonic device drops the power delivery (to the ultrasonic blade) to smoothly control the compression and coagulation of the tissue. This process has shown an ability to effectively seal vessels without transection. In this way, process 3200 may adjust 3218 the power output to the ultrasonic blade of the end effector dynamically, using a proportional-integral (PI) controller, based on a travel ratio that changes as the clamp arm approaches the ultrasonic blade. It will be appreciated that PI control is not the only logic system through which power could be controlled. Many mathematical mappings exist to appropriately reduce power as a function of the Hall-effect sensor. Examples of other logic systems include PID controllers, proportional controllers, fuzzy logic, neural networks, polynomials, Bayesian networks, among others.

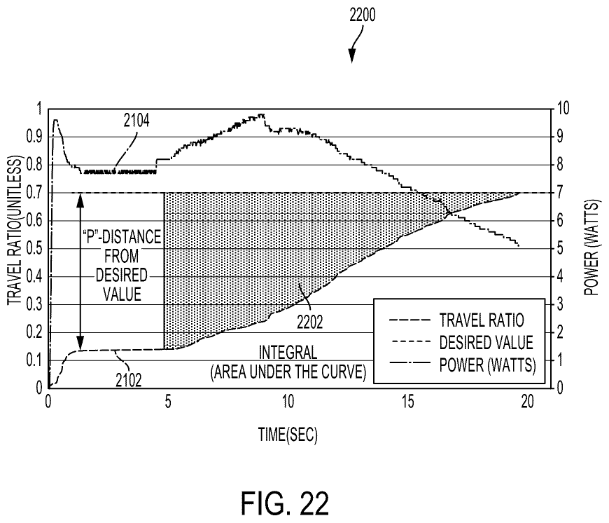

FIG. 22 is a graph 2200 depicting how the PI controller works. The Proportional term may be an indication of the absolute difference between TR and the Desired Value for TR. TR approaches the Desired Value, the effect of the proportional term may shrink and as a result the ultrasonic power (e.g., delivered by the ultrasonic blade) may be reduced. The Integral term, shown as the area 2202 under the curve, may be an accumulation of error over a give section of time. For example, as shown above, the Integral term may not begin to accumulate until after 5 seconds. After 5 seconds, the Integral term may begin to take effect and the power to the ultrasonic blade may be increased. After about 9 seconds, the reduction of the effect in the Proportional term may outweigh the effect of the increase in the Integral term causing the power delivery to the ultrasonic blade to become reduced. In this example, a Desired Value of 0.7 for the TR was used, however, as discussed above, the TR value may be optimized for a specific device along with the Proportional and Integral terms of the controller.

In effect, the PI controller may indicate what the power output should be based on the distance at any given time between the Travel Ratio and the Desired Value. From that distance, the PI controller may output a certain value (e.g., 0.4). In the example of FIG. 22, at a moment in time (e.g., 1 second) the distance is based on the values assigned to the P and I. This distance may be multiplied it by a constant that represents P and result in 0.78. The generator may instruct the system to send out 0.78 or send out, for example, 7.8 watts of power when the distance between these two is a certain amount. As a result, the TR curve approaches the Desired Value curve, and the distance reduces. Over time, the amount of power the generator tells the system to send decreases, which may be the desired outcome. However, this could also mean that if only P and not I is used, when time approaches 15 seconds, there may not be enough power output to the tissue to complete the goal. This is where the I portion (integral portion) is calculated at a set period of time, which may be about five seconds. By calculating the area 2202 under the curve (shown in FIG. 22 by hashed lines, captured between the Desired Value and Travel Ratio over time) and in addition to the 0.78, shown between 0 and 5 seconds, the I portion starts to add its own amount of power to help the progression of the Travel Ratio to the Desired Value and make sure that it gets there in a somewhat timely manner. For example, at five seconds, the I portion is not active, but as time progresses the I portion starts to calculate the area captured between the two curves and adds that value (e.g., additional four watts) which is the area under the curve, in addition to the power coming from the Proportional value. Using these two calculations together may provide the Power curve (i.e., the power output) as shown in FIG. 22. The PI controller is configured to drive towards the seal effect in a somewhat timely manner.

In one aspect the techniques described herein may be employed to seal different sizes of vessels (e.g., 5 mm, 6 mm, and 7 mm round vessels). The strength of the seals may be tested until the seal bursts and recording the burst pressure. A higher burst pressure indicates a stronger seal. In the case of an actual surgery, if a surgical instrument or device as described herein is used to seal a vessel the seal will not leak if it has a high associated burst pressure. In one aspect, the burst pressures can be measured across different sizes of vessels, e.g., 5 mm, 6 mm, and 7 mm round vessels, respectively. Typically, smaller vessels have higher burst pressure with larger vessels, the burst pressure is decreased.

Turning now to FIG. 23, there is shown several vessels 2400 that were sealed using the techniques and features described herein (e.g., using an ultrasonic blade and a Hall-effect sensor). Using the PI controller as described above, 60 vessels were sealed. 58 vessels were sealed without transection.

In one aspect, it has been observed that activating a ultrasonic blade with the clamp arm open may help to release tissue that may have stuck to the ultrasonic blade while being coagulated. Detecting a change in signal from a Hall-effect sensor may indicate when the user is opening the clamp arm after device activation. This information may trigger the system to send a low level ultrasonic signal for a short period of time, in order to release any tissue stuck to the ultrasonic blade. This short, sub-therapeutic signal may reduce the level of sticking experienced by the user. This feature may be useful if a ultrasonic shear device were designed for multiple uses and the ultrasonic blade coating began to wear off. In this way, the techniques and features described herein may be used to reduce the amount of tissue sticking to the ultrasonic blade.

A method for calibrating an end effector and Hall-effect sensor may include calibrating the end effector and Hall-effect sensor during manufacturing of thereafter. As discussed above, process 3300 shown in FIG. 32, may be used to calibrate a controller for the end effector. For example, a clamp arm position of a ultrasonic device may be calibrated during assembly. As discussed herein, sensing the position of the clamp arm relative to the ultrasonic blade may provide relevant surgical device information that may enable new capabilities, including but not limited to the ability to sense a quantity or type of tissues which may be clamped inside the jaws. Further, determinations about various algorithms to execute (e.g., such sealing a vessel without transection) may be made based on sensing the position of the clamp arm. However, in various aspects, in order for this information to be useful and reliable, the surgical device must be calibrated in relation to a baseline such as when the clamp arm is fully open or when the clamp arm fully closed with zero material in the end effector.

As described above, determining a Travel Ratio (TR) may help in various processes to control an end effector. In determining TR, X1 is the clamp arm position when the device is fully closed with no tissue. Determining the value (e.g., Hall effect signal) corresponding to X1 may be done during manufacturing and may be part of the calibration process.

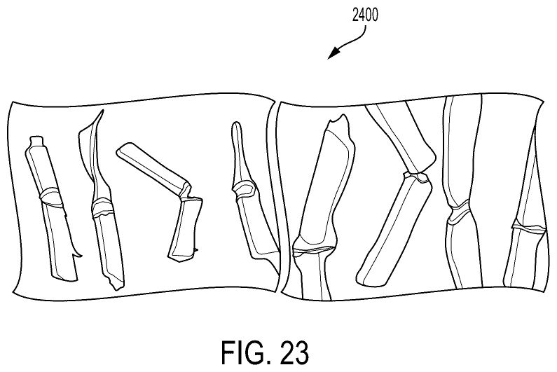

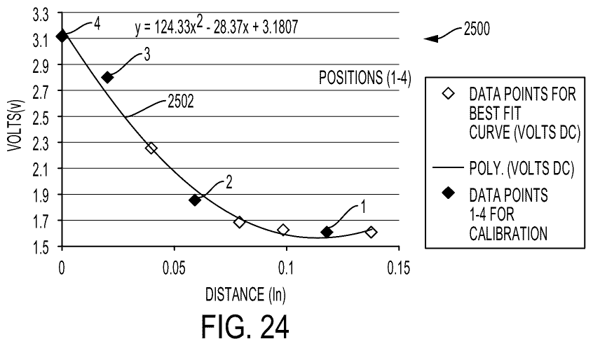

Turning now to FIG. 24, there is shown a graph 2500 of a best fit curve 2502 of Hall-effect sensor output voltage along the y-axis as a function of absolute distance (in) along the x-axis for various positions of the clamp arm. The best fit curve 2502 is plotted based on the absolute distance (in) of the clamp arm from the ultrasonic blade as listed in the third column 1406 of the table 1400 shown in FIG. 14A and the corresponding Hall-effect sensor output voltage listed in the fourth column 1408 of the table 1400 shown in FIG. 14A as the clamp arm moves from a fully open position to a fully closed position in accordance with the present disclosure.

Still with reference to FIG. 24, there is shown an example electrical output of a Hall-effect sensor configured to sense clamp arm position is shown. The Hall-effect sensor signal strength plotted against displacement of the sensor (e.g., a magnet) may follow a parabolic shape as shown by the best fit curve 2502. To calibrate the Hall-effect sensor, several readings of the sensor are taken at known baseline locations. During calibration, the best fit curve 2502 as shown in FIG. 24 may be analyzed to confirm that the Hall-effect sensor is reading effectively based on readings made in a production setting. In this way, a Hall-effect sensor response corresponding to various positions of the clamp arm (e.g., fully open, fully closed, and discrete positions therebetween) may be recorded to create a best fit curve during production. Various data points may be recorded (e.g., four data points 1-4 as shown in FIG. 24 or more as may be necessary) to create the best fit curve 2502. For example, in a first position, a Hall-effect sensor response may be measured when the clamp arm is fully opened. In this way, turning briefly to FIG. 32, the process 3300 shown in FIG. 32 may detect 3302 a first measurement signal (e.g., a Hall-effect sensor response) corresponding to a fully open position of a clamp arm and a ultrasonic blade of the end effector.

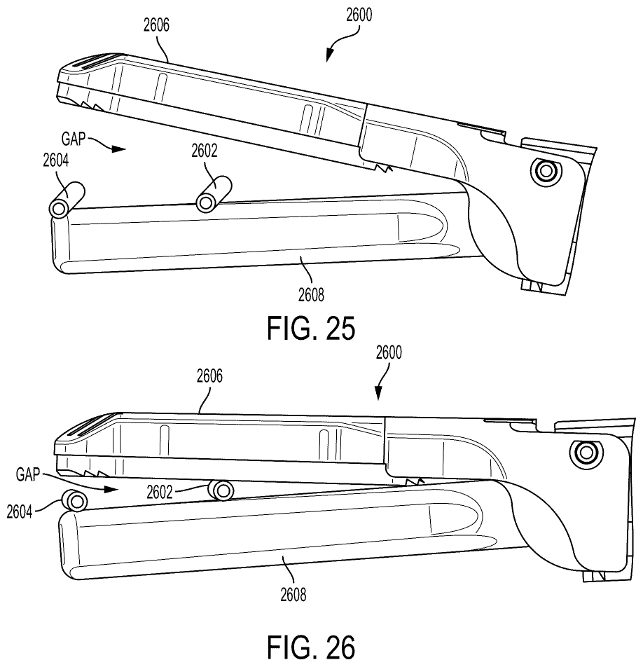

Turning back now to FIG. 24 in conjunction with FIG. 25, the four data points 1-4 represent voltage measured with a Hall-effect sensor as a function of the gap between the clamp arm 2606 and the ultrasonic blade 2608, as shown in FIG. 24. These data points 1-4 may be recorded as described in connection with FIGS. 25-28. The first data point (1) is recorded when the end effector 2600 is in the configuration shown in FIG. 25. The first data point (1) corresponds to the Hall-effect sensor output voltage recorded when the clamp arm 2606 is in the fully opened positon relative to the ultrasonic blade 2608.

The second data point (2) is recorded when the end effector 2600 is in the configuration shown in FIG. 26. To obtain an accurate gap between the clamp arm 2606 and the ultrasonic blade 2808, a first gage pin 2602 of known diameter is placed at a predetermined location within the jaws of the end effector 2600, e.g., between the clamp arm 2606 and the ultrasonic blade 2608. As shown in FIG. 26, the first gage pin 2602 is positioned between the distal end and the proximal end of the ultrasonic blade 2608 and is grasped between the clamp arm 2606 and the ultrasonic blade 2608 to set an accurate gap between the clamp arm 2606 and the ultrasonic blade 2808. Once the clamp arm 2606 is closed to grasp the first gage pin 2602, the output voltage of the Hall-effect sensor is measured and recorded. The second data point (2) is correlated to the gap set between the clamp arm 2606 and the ultrasonic blade 2608 by the first gage pin 2602. In this way, the output voltage of the Hall-effect sensor is equated to the gap distance between the clamp arm 2606 and the ultrasonic blade 2608. The second data point (2) is one of several data points to develop the polynomial to generate the best fit curve 2502 shown in FIG. 24. The process 3300 described in FIG. 32 detects 3304 an actual Hall-effect sensor voltage and determines the gap between the clamp arm 2606 and the ultrasonic blade 2608 based on the best first curve 2502 (e.g., computing the polynomial).

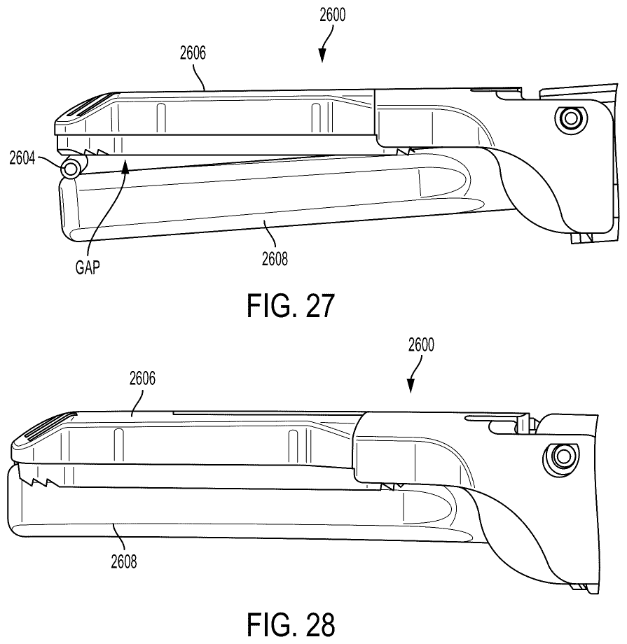

The third data point (3) is recorded when the end effector 2600 is in the configuration shown in FIG. 27. To obtain another accurate gap between the clamp arm 2606 and the ultrasonic blade 2808, the first gage pin 2602 is removed and a second gage pin 2604 of known diameter is placed at a predetermined location within the jaws of the end effector 2600, e.g., between the clamp arm 2606 and the ultrasonic blade 2608, that is different form the location of the first gage pin 2602. As shown in FIG. 27, the second gage pin 2604 is positioned between the distal end and the proximal end of the ultrasonic blade 2608 and is grasped between the clamp arm 2606 and the ultrasonic blade 2608 to set an accurate gap between the clamp arm 2606 and the ultrasonic blade 2808. Once the clamp arm 2606 is closed to grasp the second gage pin 2602, the output voltage of the Hall-effect sensor is measured and recorded. The third data point (3) is correlated to the gap set between the clamp arm 2606 and the ultrasonic blade 2608 by the second gage pin 2604. In this way, the output voltage of the Hall-effect sensor is equated to the gap distance between the clamp arm 2606 and the ultrasonic blade 2608. The third data point (3) is one of several data points to develop the polynomial to generate the best fit curve 2502 shown in FIG. 24. The process 3300 described in FIG. 32 detects 3304 an actual Hall-effect sensor voltage and determines the gap between the clamp arm 2606 and the ultrasonic blade 2608 based on the best first curve 2502 (e.g., computing the polynomial).

The fourth data point (4) is recorded when the end effector 2600 is in the configuration shown in FIG. 28. To obtain the fourth data point (4), there are no gage pins 2602, 2604 placed between the clamp arm 2606 and the ultrasonic blade 2608, but rather, the clamp arm 2606 is place in the fully closed position relative to the ultrasonic blade 2608. Once the clamp arm 2606 is placed in the fully closed position, the output voltage of the Hall-effect sensor is measured and recorded. The fourth data point (4) is correlated to the fully closed clamp arm 2606 position. In this way, the output voltage of the Hall-effect sensor is equated to the fully closed clamp arm 2606 position relative to the ultrasonic blade 2608. The fourth data point (4) is one of several data points to develop the polynomial to generate the best fit curve 2502 shown in FIG. 24. The process 3300 described in FIG. 32 detects 3304 an actual Hall-effect sensor voltage and determines the gap between the clamp arm 2606 and the ultrasonic blade 2608 based on the best first curve 2502 (e.g., computing the polynomial).

Various configurations of gage pins may create known displacements and/or angles between the clamp arm 2606 and the ultrasonic blade 2608 of the end effector 2600. Using kinematics of a given clamp arm/ultrasonic blade/shaft design and gage pins of known diameter, a theoretical displacement of the shaft assembly can be known at each of the, e.g., four or more positions. This information may be input, along with the voltage readings of the Hall-effect sensor, to fit a parabolic curve (e.g., best fit curve 2502 as shown in FIG. 24), which may become a characteristic of each individual surgical device. This information may be loaded onto the surgical device via an EEPROM or other programmable electronics configured to communicate with the generator (e.g., the generator 102 shown in FIG. 6) during use of the surgical device.

The Hall-effect sensor signal response at, for example, the four positions of the clamp arm described above may be graphed and the responses may be fit and entered into to a lookup table or developed into a polynomial which may be used to set/calibrate the Hall-effect sensor such that when used by a surgeon, the end effector delivers the tissue effect desired. In this way, process 3300 may determine 3308 a best fit curve to represent signal strength (e.g., from Hall-effect sensor) as a function of sensor displacement (e.g., magnet displacement) based on at least the first, second, and third signals, the fully open, intermediate, and fully closed positions, and a dimension of the rigid body. Process 3300 may also create 3310 a lookup table based on at least the first, second, and third signals, and the fully open, intermediate, and fully closed positions.

The positioning of the Hall-effect sensor/magnet arrangement in the configurations described above may be used to calibrate the surgical device such that the most sensitive movements of the clamp arm 2606 exist when the clamp arm 2606 is closest to the ultrasonic blade 2608. Four positions, corresponding to four data points (1-4), were chosen in the example described above, but any number of positions could be used at the discretion of design and development teams to ensure proper calibration.

In one aspect, the techniques and features described herein may be used to provide feedback to a surgeon to indicate when the surgeon should use hemostasis mode for the vessel sealing procedure prior to engaging the cutting procedure. For example, hemostasis mode algorithm may be dynamically changed based on the size of a vessel grasped by the end effector 2600 in order to save time. This may require feedback based on the position of the clamp arm 2606.

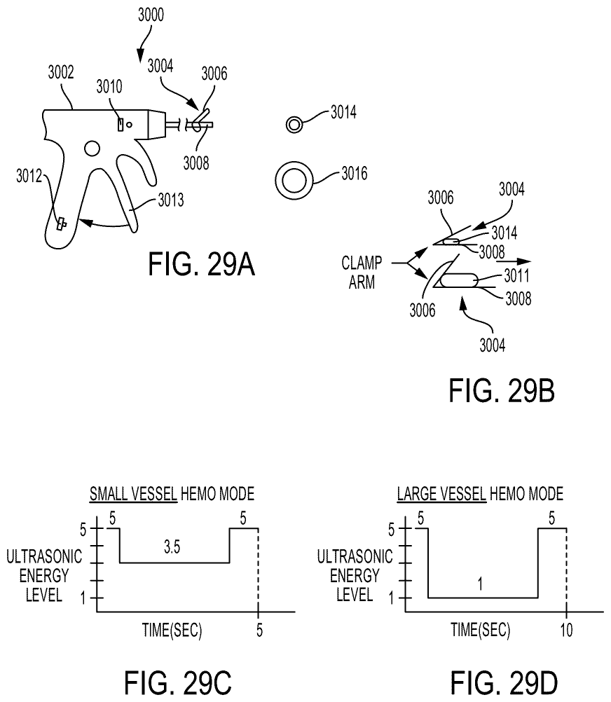

FIG. 29A is a schematic diagram 3000 of surgical instrument 3002 configured to seal small and large vessels in accordance with one aspect of the present disclosure. The surgical instrument 3002 comprises an end effector 3004, where the end effector comprises a clamp arm 3006 and an ultrasonic blade 3008 for treating tissue including vessels of various sizes. The surgical instrument 3002 comprises a Hall-effect sensor 3010 to measure the position of the end effector 3004. A closure switch 3012 is provided to provide a feedback signal indicating whether the trigger handle 3013 of the surgical instrument is in a fully closed position.

Turning now to FIG. 29B there is shown a diagram of an example range of a small vessel 3014 and a large vessel 3016 and the relative position of a clamp arm of the end effector in accordance with one aspect of the present disclosure. With reference to FIGS. 29A-B, The surgical instrument 3002 shown in FIG. 29A is configured to seal small vessels 3014 having a diameter <4 mm and large vessels 3016 having a diameter >4 mm and the relative position of the clamp arm 3006 when grasping small and large vessels 3014, 3016 and the different voltage readings provided by the end effector 3010 depending on the size of the vessel.

FIGS. 29C and 29D are two graphs 3020, 3030 that depicts two processes for sealing small and large vessels by applying various ultrasonic energy levels for a different periods of time in accordance with one aspect of the present disclosure. Ultrasonic energy level is shown along the y-axis and time (Sec) is shown along the x-axis. With reference now to FIGS. 29A-C, the first graph 3020 shown in FIG. 29C shows a process for adjusting the ultrasonic energy drive level of a ultrasonic blade to seal a small vessel 3014. In accordance with the process illustrated by the first graph 3020 for sealing and transecting a small vessel 3014, a high ultrasonic energy (5) is applied for a first period 3022. The energy level is then lowered to (3.5) for a second period 3024. Finally, the energy level is raised back to (5) for a third period 3026 to complete sealing the small vessel 3014 and achieve transection and then the energy level is turned off. The entire cycle lasting about 5 seconds.

With reference now to FIGS. 29A-D, the second graph 3030 shown in FIG. 29D shows a process for adjusting the ultrasonic energy drive level of a ultrasonic blade to seal a large vessel 3016. In accordance with the process illustrated by the second graph 3030 for sealing and transecting a large vessel 3016, a high ultrasonic energy (5) is applied for a first period 3032. The energy level is then lowered to (1) for a second period 3034. Finally, the energy level is raised back to (5) for a third period 3036 to complete sealing the large vessel 3016 and achieve transection and then the energy level is turned off. The entire cycle lasting about 10 seconds.

Smaller vessels 3014 may be easier to seal at high burst pressure levels. Thus, it may be desirable to sense and determine whether a smaller vessel 3014 (e.g., less than 4 mm) is clamped by the clamp arm 3006, and if so, the ultrasonic energy level may not need to be dropped to 1. Instead, the energy level could be dropped less, to about 3.5 for example, as shown by the first graph 3020 shown in FIG. 29C. This may allow the surgeon to get through the vessel, coagulate it, and cut the vessel more quickly with knowledge that the process can go faster because the vessel 3014 is slightly smaller. If the vessel 3016 is larger, (e.g., 4 mm or greater) a process that heats the vessel slower and over a longer period of time may be more desirable, as shown by the second graph 3030 in FIG. 29D.

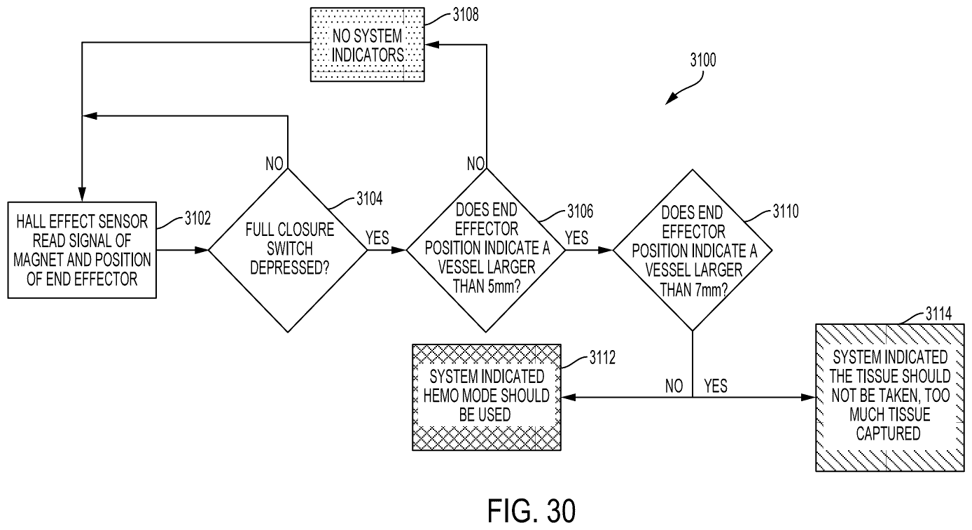

FIG. 30 is a logic diagram illustrating an example process 3100 for determining whether hemostasis mode should be used, in accordance with one aspect of the present disclosure. At the outset, the process 3100 reads 3102 the signal from a Hall-effect sensor determine 3102 the position of an end effector. The process 3100 then determines 3104 if a full closure switch of the surgical device is depressed, or if the handle of the surgical device is fully closed. If the full closure switch of the surgical device is not depressed and/or if the handle of the surgical device is not fully closed, the process 3100 may continue to read 3102 the Hall-effect sensor to determine the position of the end effector. If the full closure switch of the surgical device is depressed, or if the handle of the surgical device is fully closed, the process 3100 determines 3106 if the end effector position indicates a vessel larger than 5 mm. If the end effector position does not indicate a vessel larger than 5 mm, and no system indicators are found 3108, the process 3100 may continue to read 3102 the Hall-effect sensor and determine the position of the end effector.

If the end effector position indicates a vessel larger than 5 mm, the process 3100 determines 3110 if the end effector position indicates a vessel larger than 7 mm. If the end effector position does not indicate a vessel larger than 7 mm, the process 3100 indicates 3112 that hemostasis mode should be used. This condition may be indicated using a variety of auditory, vibratory, or visual feedback techniques including, for example, a green LED located on the surgical device (e.g., on top of the handle) may be enabled. If the end effector position does indicate a vessel larger than 7 mm, the process 3100 indicates 3114 that the tissue should not be taken (i.e., hemostasis mode should not be used) because too much tissue has been captured by the end effector. This condition may be indicated using a variety of auditory, vibratory, or visual feedback techniques including, for example, a red LED on the surgical device (e.g., on top of the handle) may be enabled.

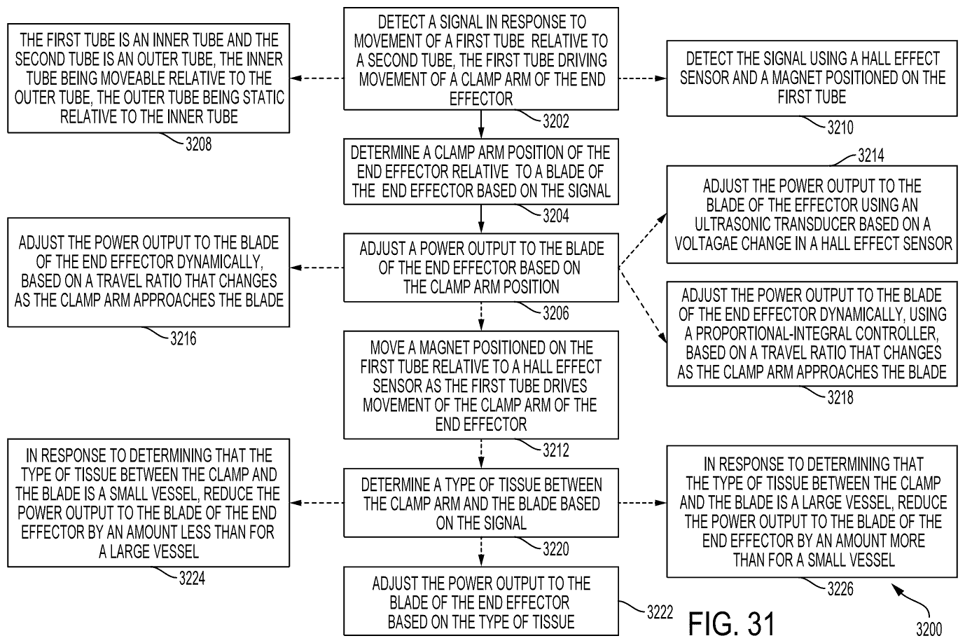

FIG. 31 is a logic diagram illustrating an example process 3200 for end effector control in accordance with one aspect of the present disclosure. In one aspect Referring to FIG. 31, process 3200 detects 3202 a signal (e.g., at a Hall-effect sensor) in response to movement of a first tube relative to a second tube, the first tube driving movement of a clamp arm of the end effector. The first tube may be, for example, similar to reciprocating tubular actuating member 58 (FIGS. 3 and 4) and the second tube may be, for example, similar to outer tubular sheath 56 (FIGS. 3 and 4). In other words, as described in FIG. 31, the first tube may be an inner tube and the second tube is an outer tube. The inner tube may be moveable 3208 relative to the outer tube. The outer tube may be static relative to the inner tube. The process 3200 detects 3210 the signal using a Hall-effect sensor and a magnet positioned on the first tube.

The process 3200 continues and determines 3204 a clamp arm position of the end effector relative to a ultrasonic blade of the end effector based on the signal (from, e.g., Hall-effect sensor voltage output). Once clamp arm position relative to ultrasonic blade is known, the vibrational mode of the ultrasonic blade can be adjusted to obtain different tissue effects. In this way, the process 3200 adjusts 3206 a power output to the ultrasonic blade of the end effector based on the clamp arm position. For example, the process 3200 may adjust 3214 the power output to the ultrasonic blade of the end effector using an ultrasonic transducer based on a voltage change in a Hall-effect sensor. Alternatively, the process can effectively seal vessels without transection. In this way, the process 3200 may adjust 3218 the power output to the ultrasonic blade of the end effector dynamically, using a proportional-integral controller, based on a travel ratio that changes as the clamp arm approaches the ultrasonic blade.

In another aspect, the process 3200 may adjust 3216 the power output to the ultrasonic blade of the end effector dynamically, based on the travel ratio that changes as the clamp arm approaches the ultrasonic blade. For example, as the clamp arm moves towards the ultrasonic blade and a Desired Value (FIGS. 21 and 22) is approached, the amount of power output to the ultrasonic blade and into the tissue may be reduced. This is because the ultrasonic blade will cut the tissue with enough power. However, if the power being output is reduced over time as the Desired Value is approached (where a full transection may be represented by a Travel Ratio of 1), the chance that the tissue is transected may be drastically reduced. In this way, effective sealing may be achieved without cutting the tissue as may be desired by the surgeon.

In one aspect, the process 3200 of FIG. 31 moves 3212 a magnet positioned on the first tube relative to a Hall-effect sensor as the first tube drives movement of the clamp arm of the end effector. The process 3200 then determines 3220 a type of tissue between the clamp arm and the ultrasonic blade based on a signal (from, e.g., a Hall-effect sensor). Further, using a processor and/or memory, one or more algorithms (e.g., for sealing a vessel without transection) may be chosen based on the thickness, quantity, or type of tissue determined to be clamped inside the jaws. In response to determining that the type of tissue between the clamp and the ultrasonic blade is a large vessel, the process 3200 may reduce 3226 the power output to the ultrasonic blade of the end effector by an amount more than for a small vessel. Further, in response to determining that the type of tissue between the clamp and the ultrasonic blade is a small vessel, the process 3200 may reduce 3224 the power output to the ultrasonic blade of the end effector by an amount less than for a large vessel. In one aspect, instead of changing algorithms for small vessels as described above, an indicator may be provided to the surgeon to indicate the thickness of the tissue captured in the end effector. In one aspect, the process 3200 adjusts 3222 the power output to the ultrasonic blade of the end effector based on the type of tissue.

FIG. 32 is a logic diagram illustrating an example process 3300 for calibrating an apparatus for controlling for an end effector in accordance with one aspect of the present disclosure. In one aspect, the process 3300 detects 3302 a first signal corresponding to a fully open position of a clamp arm and a blade of the end effector. The process 3300 then detects 3304 a second signal corresponding to an intermediate position of the clamp arm and the blade of the end effector, the intermediate position resulting from clamping a rigid body between the clamp arm and the blade. The process detects 3306 a third signal corresponding to a fully closed position of the clamp arm and the blade of the end effector. Once the three signals are detected, the process 3300 determines 3308 a best fit curve to represent signal strength as a function of sensor displacement based on at least the first, second, and third signals corresponding to the fully open, intermediate, and fully closed positions, respectively, and a dimension of the rigid body. An example of a best fit curve in this context is shown in FIGS. 14B and 24. Finally, the process 3300 creates 3310 a lookup table based on at least the first, second, and third signals corresponding to the fully open, intermediate, and fully closed positions, respectively.

As described hereinabove, the position of the clamp arm portion of the end effector can be measured with a Hall-effect sensor/magnet arrangement. A tissue pad, usually made of TEFLON, may be positioned on the clamp arm to prevent tissue from sticking to the clamp arm. As the end effector is used and the tissue pad is worn, it will be necessary to track the drift of the Hall-effect sensor output signal and establish changing thresholds to maintain the integrity of the tissue treatment algorithm selection and the end of cut trigger points feedback to the tissue treatment algorithms.

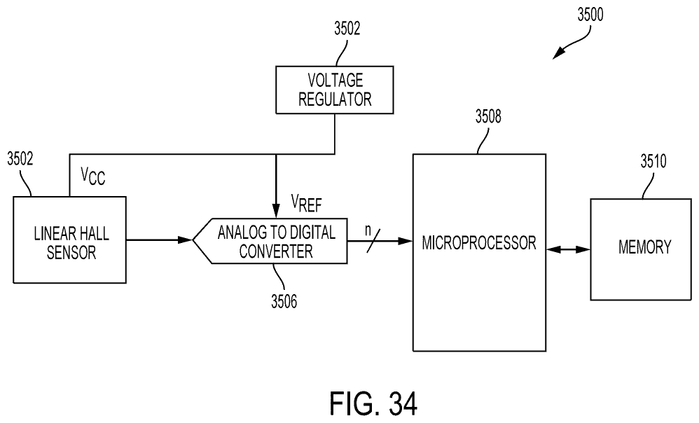

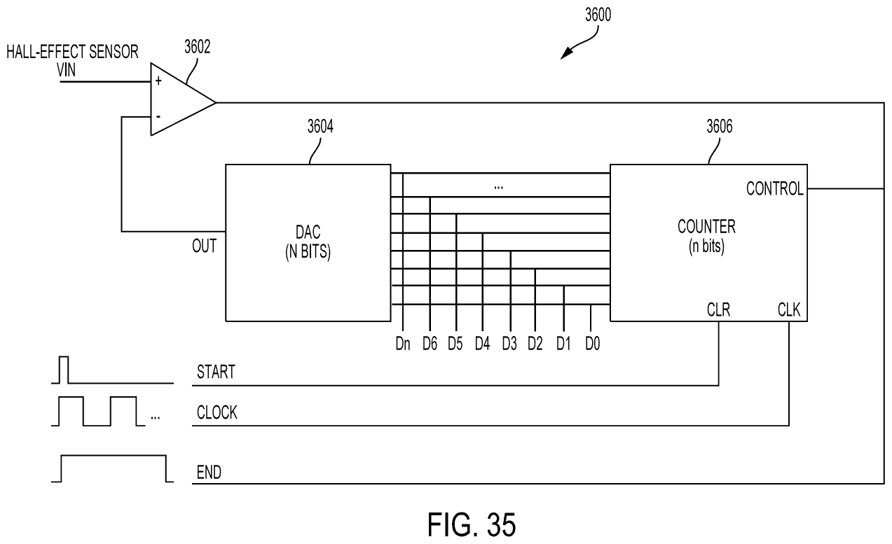

Accordingly, a control system is provided. The output of the Hall-effect sensor in the form of counts can be used to track the aperture of the end effector clamp arm. The reader may refer to FIGS. 34 and 35 for ADC systems 3500, 3600 that can employ the counter output of an ADC. The clamp arm position, with or without a tissue pad, can be calibrated using the techniques described herein. Once the clamp arm position is calibrated, the position of the clamp arm and wear of the tissue pad can be monitored. In one aspect, the control system determines that the clamp arm is in a closed position by monitoring for a rise in acoustic impedance that occurs when the ultrasonic blade contacts either tissue or the tissue pad. Thus, a specific number of ADC counter will accumulate a specific number of counts from the time the clamp arm goes from a fully open position to a fully closed position. In one implementation, based on the configuration of the Hall-effect sensor, the Hall-effect sensor ADC counts increase as the clamp arm closes towards the ultrasonic blade. As the tissue pad wears, the counter will accumulate an incremental additional number of counts due to the additional rotational travel experienced by the clamp arm due to tissue pad wear. By tracking the new count value for a closed clamp arm position, the control system can adjust the trigger threshold for an end of cut and better predict the total range of aperture of the clamp arm that has occurred.

Furthermore, the Hall-effect sensor ADC counts may be employed to determine the tissue coefficient of friction (u) of the tissue under treatment based on the aperture of the clamp arm by employing predetermined u values stored in a look-up table. For example, the specific tissue treatment algorithm can be dynamically adjusted or changed during an ultrasonic treatment cycle (e.g., firing sequence or activation of ultrasonic energy) to optimize the tissue cut based on the tissue type (e.g., fatty tissue, mesentery, vessel) or the tissue quantity or thickness.

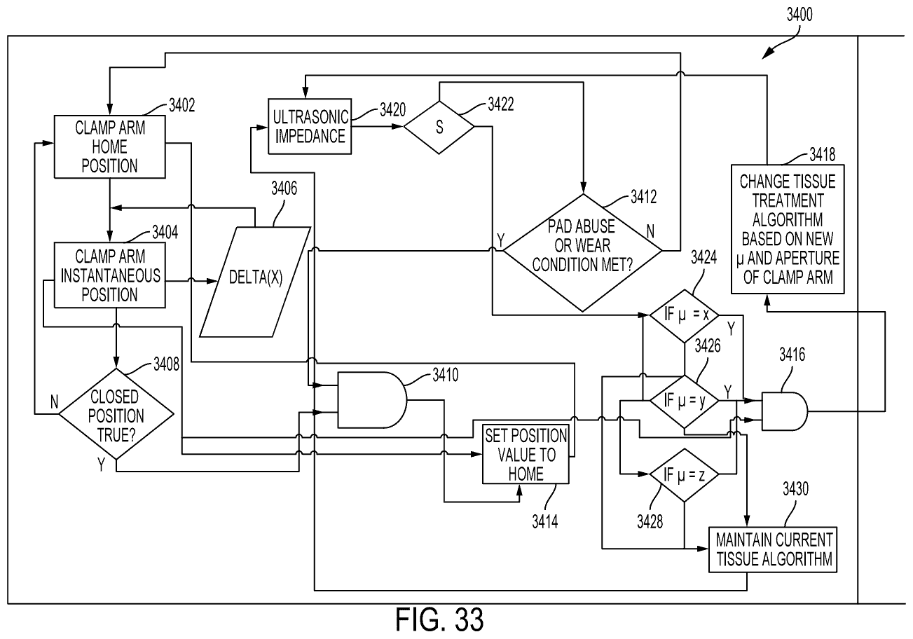

FIG. 33 is a logic diagram of a process 3400 for tracking wear of the tissue pad portion of the clamp arm and compensating for resulting drift of the Hall-effect sensor and determining tissue coefficient of friction, according to one aspect of the present disclosure. The process 3400 may be implemented in software, hardware, firmware, or a combination thereof, employing the generator circuit environment illustrated in connection with FIGS. 6-10.

In one aspect, the process 3400 may be implemented by a circuit may comprising a controller comprising one or more processors (e.g., microprocessor, microcontroller) coupled to at least one memory circuit. The at least one memory circuit stores machine executable instructions that when executed by the processor, cause the processor to execute the process 3400.

The processor may be any one of a number of single or multi-core processors known in the art. The memory circuit may comprise volatile and non-volatile storage media. In one aspect, the processor may include an instruction processing unit and an arithmetic unit. The instruction processing unit may be configured to receive instructions from the one memory circuit.

In one aspect, a circuit may comprise a finite state machine comprising a combinational logic circuit configured to implement the process 3400 described herein. In one aspect, a circuit may comprise a finite state machine comprising a sequential logic circuit comprising a combinational logic circuit and at least one memory circuit, for example. The at least one memory circuit can store a current state of the finite state machine. The sequential logic circuit or the combinational logic circuit can be configured to implement the process 3400 described herein. In certain instances, the sequential logic circuit may be synchronous or asynchronous.

In other aspects, the circuit may comprise a combination of the processor and the finite state machine to implement the compression and decompression techniques described herein. In other embodiments, the finite state machine may comprise a combination of the combinational logic circuit and the sequential logic circuit.

As described herein, the position of the clamp arm is sensed by a Hall-effect sensor relative to a magnet located in a closure tube of a surgical instrument. Turning now to the process 3400, the initial home position of the clamp arm, e.g., the position of the Hall-effect sensor located on the closure tube, is stored 3402 in memory. As the closure tube is displaced in a distal direction, the clamp arm is closed towards the ultrasonic blade and the instantaneous position of the clamp arm is stored 3404 in memory. The difference, delta (x), between the instantaneous position and the home position of the clamp arm is calculated 3406. The difference, delta (x), may be used to determine a change in displacement of the tube, which can be used to calculate the angle and the force applied by the clamp arm to the tissue located between the clamp arm and the ultrasonic blade. The instantaneous position of the clamp arm is compared 3408 to the closed position of the clamp arm determine whether the clamp arm is in a closed position. While the clamp arm is not yet in a closed position, the process 3400 proceeds along the no path (N) and compares the instantaneous position of the clamp arm with the home position of the clamp arm until the clamp reaches a closed position.

When the clamp arm reaches a closed position, the process 3400 continues along the yes path (Y) and the closed position of the clamp arm is applied to one input of a logic AND function 3410. The logic AND function 3410 is a high level representation of a logic operation, which may comprise boolean AND, OR, XOR, and NAND operations implemented either in software, hardware, or a combination thereof. When a tissue pad abuse or wear condition is determined based on acoustic impedance measurements, the current clamp arm closed position is set 3414 as the new home position of the clamp arm to compensate for the abuse or wear condition. If no tissue pad abuse or wear is determined, the home position of the clamp arm remains the same. Abuse or wear of the clamp arm tissue pad is determined by monitoring 3420 the impedance 3422 of the ultrasonic blade. The tissue pad/ultrasonic blade interface impedance id s determined 3422 and compared 3412 to a tissue pad abuse or wear condition. When the impedance corresponds to a tissue pad abuse or wear condition, the process 3400 proceeds along the yes path (Y) and the current closed position of the clamp arm is set 3414 as the new home position of the clamp arm to compensate for the abuse or wear condition of the tissue pad. When the impedance does not correspond to a tissue pad abuse or wear condition, the process 3400 proceeds along the no path (N) and the home position of the clamp arm remains the same.

The stored 3404 instantaneous position of the clamp arm is also provided to the input of another logic AND function 3416 to determine the quantity and thickness of the tissue clamped between the clamp arm and the ultrasonic blade. The tissue/ultrasonic blade interface impedance is determined 3422 and is compared 3424, 34267, 3428 to multiple tissue coefficients of friction μ=x, μ=y, or μ=z. Thus, when the tissue/ultrasonic blade interface impedance corresponds to one of the tissue coefficients of friction μ=x, μ=y, or μ=z based on the tissue quantity or thickness, e.g., the aperture of the clamp arm, the current tissue algorithm is maintained 3430 and the current algorithm is used for monitoring 3420 the impedance 3422 of the ultrasonic blade. If the tissue coefficient of friction μ=x, μ=y, or μ=z based on the tissue quantity or thickness, e.g., the aperture of the clamp arm, changes, the current tissue algorithm is changed 3418 based on the new tissue coefficient of friction u and the tissue quantity or thickness, e.g., the aperture of the clamp arm and the new algorithm is used for monitoring 3420 the impedance 3422 of the ultrasonic blade.

Accordingly, the current clamp arm aperture is used to determine the current tissue coefficient of friction u based on the quantity and thickness of tissue as measured by the aperture of the clamp arm. Thus, an initial algorithm may be based on an initial aperture of the clamp arm. The impedance of the ultrasonic blade is compared 3424, 3426, 3428 to several tissue coefficients of friction μ=x, μ=y, or μ=z, which are stored in a look-up table, and correspond to fatty tissue, mesentery tissue, or vessel tissue, for example. If no match occurs between the impedance of the ultrasonic blade and the tissue coefficient of friction, the process 3400 proceeds along the no paths (N) of any of the tissue impedance comparisons 3424, 3426, 3428 and the current tissue algorithm is maintained. If any one of the outputs of the comparison 3424, 3426, 3428 functions is true, the processor switches to a different tissue treatment algorithm based on the new tissue impedance and clamp arm aperture. Accordingly, a new tissue treatment algorithm is loaded in the ultrasonic instrument. The process 3400 continues by monitoring 3420 the impedance of the ultrasonic blade, clamp arm aperture, and tissue pad abuse or wear.

FIG. 34 illustrates a Hall-effect sensor system 3500 that can be employed with the process 3400 of FIG. 33, according to one aspect of the present disclosure. In connection with the process 3400 described in FIG. 33, the Hall-effect sensor system 3500 of FIG. 34 includes a Hall-effect sensor 3502 powered by a voltage regulator 3504. The output of the Hall-effect sensor 3502 is an analog voltage proportional to the position of the clamp arm, which is applied to an analog-to-digital converter 3506 (ADC). The n-bit digital output of the ADC 3506 is applied to a microprocessor 3508 coupled to a memory 3510. The microprocessor 3508 is configured to process and determine the position of the clamp arm based on the n-bit digital input from the ADC 3505. It will be appreciated that the digital output of the ADC 3506 may be referred to as a count.

As described herein, the analog output of the Hall-effect sensor is provided to an internal or an external analog-to-digital converter such as the ADC 3506 shown in FIG. 34 or any of the analog-to-digital converter circuits located in the generator. The transducer 104 shown in FIG. 6 may comprise an Hall-effect sensor comprising and analog-to-digital converter circuit whose output is applied to the control circuit 108. In one aspect, the generator 102 shown in FIG. 7 comprises several analog-to-digital converter circuits such as ADCs 176, 178, 180, which can be adapted and configured to receive the analog voltage output of the Hall-effect sensor and convert it into digital forms to obtain counts and to interface the Hall-effect sensor with a DSP processor 174, microprocessor 190, a logic device 166, and/or a controller 196.

FIG. 35 illustrates one aspect of a ramp type counter analog-to-digital converter 3600 (ADC) that may be employed with the Hall-effect sensor system 3500 of FIG. 34, according to one aspect of the present disclosure. The digital ramp ADC 3600 receives an analog input voltage from a Hall-effect sensor at the Vin positive input terminal of a comparator 3602 and Dn through D0 (Dn-D0) are the digital outputs (n-bits). The control line found on a counter 3606 turns on the counter 3606 when it is low and stops the counter 3606 when it is high. In operation, the counter 3606 is increased until the value found on the counter 3606 matches the value of the analog input signal at Vin. The digital output Dn-D0 is applied to a digital-to-analog converter 3604 (DAC) and the analog output is applied to the negative terminal of the comparator 3602 and it is compared to the analog input voltage at Vin. When this condition is met, the value on the counter 3606 is the digital equivalent of the analog input signal at Vin.

A START pulse is provided for each analog input voltage Vin to be converted into a digital signal. The END signal represents the end of the conversion for each individual analog input voltage found at Vin (each sample), and not for the entire analog input signal. Each clock pulse increments the counter 3606. Supposing an 8-bit ADC, for converting the analog value for “128” into digital, for example, it would take 128 clock cycles. The ADC 3600 counts from 0 to the maximum possible value (2n−1) until the correct digital output Dn-D0 value is identified for the analog input voltage present at Vin. When this is true, the END signal is given and the digital value for Vin is for at Dn-D0.

While various aspects have been described herein, it should be apparent, however, that various modifications, alterations and adaptations to those aspects may occur to persons skilled in the art with the attainment of some or all of the advantages of the invention. The disclosed aspects are therefore intended to include all such modifications, alterations and adaptations without departing from the scope and spirit of the invention. Accordingly, other aspects and implementations are within the scope of the following claims. For example, the actions recited in the claims can be performed in a different order and still achieve desirable results.

While various details have been set forth in the foregoing description, it will be appreciated that the various aspects of the techniques for operating a generator for digitally generating electrical signal waveforms and surgical instruments may be practiced without these specific details. One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Further, while several forms have been illustrated and described, it is not the intention of the applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

For conciseness and clarity of disclosure, selected aspects of the foregoing disclosure have been shown in block diagram form rather than in detail. Some portions of the detailed descriptions provided herein may be presented in terms of instructions that operate on data that is stored in one or more computer memories or one or more data storage devices (e.g. floppy disk, hard disk drive, Compact Disc (CD), Digital Video Disk (DVD), or digital tape). Such descriptions and representations are used by those skilled in the art to describe and convey the substance of their work to others skilled in the art. In general, an algorithm refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

Unless specifically stated otherwise as apparent from the foregoing disclosure, it is appreciated that, throughout the foregoing disclosure, discussions using terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. In one form, several portions of the subject matter described herein may be implemented via an application specific integrated circuits (ASIC), a field programmable gate array (FPGA), a digital signal processor (DSP), or other integrated formats. However, those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In some instances, one or more elements may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but yet still co-operate or interact with each other. It is to be understood that depicted architectures of different components contained within, or connected with, different other components are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated also can be viewed as being “operably connected,” or “operably coupled,” to each other to achieve the desired functionality, and any two components capable of being so associated also can be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components, and/or electrically interacting components, and/or electrically interactable components, and/or optically interacting components, and/or optically interactable components.

In other instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present disclosure have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “one form,” or “a form” means that a particular feature, structure, or characteristic described in connection with the aspect is included in at least one aspect. Thus, appearances of the phrases “in one aspect,” “in an aspect,” “in one form,” or “in an form” in various places throughout the specification are not necessarily all referring to the same aspect. Furthermore, the particular features, structures or characteristics may be combined in any suitable manner in one or more aspects.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

All of the above-mentioned U.S. patents, U.S. patent application publications, U.S. patent applications, foreign patents, foreign patent applications, non-patent publications referred to in this specification and/or listed in any Application Data Sheet, or any other disclosure material are incorporated herein by reference, to the extent not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

Various aspects of the subject matter described herein are set out in the following numbered clauses:

1. A method for controlling an end effector, the method comprising: detecting a signal in response to movement of a first tube relative to a second tube, the first tube driving movement of a clamp arm of the end effector; determining a clamp arm position of the end effector relative to a ultrasonic blade of the end effector based on the signal; and adjusting a power output to the ultrasonic blade of the end effector based on the clamp arm position.

2. The method of clause 1, wherein adjusting the power output to the ultrasonic blade is achieved by manipulating the electrical current sent to the handpiece.

3. The method of clause 1 or 2, wherein the first tube is an inner tube and the second tube is an outer tube, the inner tube being moveable relative to the outer tube, the outer tube being static relative to the inner tube.

4. The method of any one of clause 1 or 2, wherein the first tube is an inner tube and the second tube is an outer tube, the outer tube being moveable relative to the inner tube, the inner tube being static relative to the inner tube.

5. The method of any one of clauses 1-4 further comprising detecting the signal using a Hall-effect sensor and a magnet positioned on the first tube.

6. The method of any one of clauses 1-5, further comprising moving a magnet positioned on the first tube relative to a Hall-effect sensor as the first tube drives movement of the clamp arm of the end effector.

7. The method of any one of clauses 1-6, further comprising adjusting the power output to the ultrasonic blade of the end effector using an ultrasonic transducer based on a voltage change in a Hall-effect sensor.

8. The method of any one of clauses 1-7, further comprising adjusting the power output to the ultrasonic blade of the end effector dynamically, based on a travel ratio that changes as the clamp arm approaches the ultrasonic blade.

9. The method of any one of clauses 1-8, further comprising adjusting the power output to the ultrasonic blade of the end effector dynamically, using a proportional-integral controller, based on a travel ratio that changes as the clamp arm approaches the ultrasonic blade.

10. The method of any one of clauses 1-9, further comprising switching off completely the power output to the ultrasonic blade of the end effector once a travel ratio threshold has been met.

11. The method of any one of clauses 1-10, further comprising: determining a quantity or thickness of tissue between the clamp arm and the ultrasonic blade based on the signal; and
adjusting the power output to the ultrasonic blade of the end effector based on the quantity or thickness of tissue.

12. The method of clause 11, further comprising in response to determining that the quantity or thickness of tissue between the clamp arm and the ultrasonic blade is less than a predetermined threshold, reducing the power output to the ultrasonic blade of the end effector by an amount less than for a larger quantity or thickness of tissue.

13. The method of clause 11 or 12, further comprising in response to determining that the quantity or thickness of tissue between the clamp arm and the ultrasonic blade is above a predetermined threshold, reducing the power output to the ultrasonic blade of the end effector by an amount more than for a smaller quantity or thickness of tissue.

14. An apparatus for controlling an end effector, the apparatus comprising: a sensor configured to detect a signal in response to movement of a first tube relative to a second tube, the first tube driving movement of a clamp arm of the end effector; a processor configured to determine a clamp arm position of the end effector relative to a ultrasonic blade of the end effector based on the signal; and a transducer configured to adjust a power output to the ultrasonic blade of the end effector based on the clamp arm position.

15. The apparatus of clause 14, wherein the first tube is an inner tube and the second tube is an outer tube, the outer tube being moveable relative to the inner tube, the inner tube being static relative to the outer tube.

16. The apparatus of clause 14, wherein the first tube is an inner tube and the second tube is an outer tube, the inner tube being moveable relative to the outer tube, the outer tube being static relative to the inner tube.

17. The apparatus of any one of clauses 14-16, further comprising: a magnet positioned on the first tube; and wherein the sensor is a Hall-effect sensor used to detect the signal based on a position of the magnet.

18. The apparatus of any one of clauses 14-17, wherein the magnet positioned on the first tube moves relative to a Hall-effect sensor as the first tube drives movement of the clamp arm of the end effector.

19. The apparatus of any one of clauses 14-18, wherein the transducer is an ultrasonic transducer configured to adjust the power output to the ultrasonic blade of the end effector based on a voltage change in a Hall-effect sensor.

20. The apparatus of any one of clauses 14-19, wherein the transducer is configured to adjust the power output to the ultrasonic blade of the end effector dynamically, based on a travel ratio that changes as the clamp arm approaches the ultrasonic blade.

21. The apparatus of any one of clauses 14-20, further comprising: a proportional-integral controller configured to adjust the power output to the ultrasonic blade of the end effector dynamically, based on a travel ratio that changes as the clamp arm approaches the ultrasonic blade.

22. A method for calibrating an apparatus for controlling an end effector, the method comprising: detecting a first signal corresponding to a fully open position of a clamp arm and a ultrasonic blade of the end effector; detecting a second signal corresponding to an intermediate position of the clamp arm and the ultrasonic blade of the end effector, the intermediate position resulting from clamping a rigid body between the clamp arm and the ultrasonic blade; and detecting a third signal corresponding to a fully closed position of the clamp arm and the ultrasonic blade of the end effector.

23. The method of clause 22, further comprising: determining a best fit curve to represent signal strength as a function of sensor displacement based on at least the first, second, and third signals, the fully open, intermediate, and fully closed positions, and a dimension of the rigid body.

24. The method of clause 22 or 23, further comprising: creating a lookup table based on at least the first, second, and third signals, and the fully open, intermediate, and fully closed positions.

1-24. (canceled)

25. An apparatus, comprising:
an end effector comprising an ultrasonic blade and a clamp arm pivotally movable between a plurality of positions relative to the ultrasonic blade;
a sensor to generate a signal based on a movement of the clamp arm relative to the ultrasonic blade; and
a control circuit to:
receive the signal generated by the sensor;
determine a position of the clamp arm from the plurality of positions relative to the ultrasonic blade based on the signal; and
adjust an operating parameter associated with the ultrasonic blade based on the position of the clamp arm.

26. The apparatus of claim 25, further comprising a first tube movable relative to a second tube, wherein movement of the first tube relative to the second tube causes the movement of the clamp arm relative to the ultrasonic blade.

27. The apparatus of claim 26, further comprising a magnet positioned on the first tube, wherein the signal comprises a voltage change based on a motion of the magnet relative to the sensor.

28. The apparatus of claim 25, wherein determination of the position of the clamp arm is further based on a fit curve of voltage change as a function of the plurality of positions relative to the ultrasonic blade.

29. The apparatus of claim 28, wherein the control circuit is further to:
calibrate signals generated by the sensor based on the fit curve; and
confirm that the sensor is generating accurate signals based on the calibration.

30. The apparatus of claim 25, wherein adjustment of the operating parameter is further based on waveform samples stored in a dynamically-updated lookup table.

31. The apparatus of claim 25, wherein the operating parameter comprises at least one of a waveform shape, a frequency, or an amplitude of a drive signal output by a generator, or combinations thereof.

32. The apparatus of claim 25, further comprising a feedback device, and wherein the control circuit is further to cause the feedback device to provide an indication associated with the adjusted operating parameter.

33. The apparatus of claim 32, wherein the feedback device comprises a speaker, and wherein the indication comprises a sound associated with the adjusted operating parameter.

34. The apparatus of claim 32, wherein the feedback device comprises a display, and wherein the indication comprises a light associated with the adjusted operating parameter.

35. The apparatus of claim 32, wherein the control circuit is to further delay adjustment of the operating parameter of the ultrasonic blade after causing the feedback device to provide the indication

36. The apparatus of claim 25, further comprising a ratcheting device to generate an audible sound associated with operating parameters of the ultrasonic blade.

37. The apparatus of claim 25, wherein the control circuit is further to prevent power from being delivered to the end effector in excess of a predetermined threshold.

38. An method, comprising:
moving a clamp arm of an end effector relative to an ultrasonic blade of the end effector;
generating, via a sensor, a signal based on the movement of the clamp arm of the end effector relative to the ultrasonic blade of the end effector;
receiving, via a control circuit, the signal generated by the sensor;
determining, via the control circuit, a position of the clamp arm relative to the ultrasonic blade based on the signal; and
adjusting, via the control circuit, an operating parameter associated with the ultrasonic blade based on the clamp arm position.

39. The method of claim 38, wherein determining the position of the clamp arm comprises:
referencing, via the control circuit, a fit curve of voltage change as a function of a plurality of positions relative to the ultrasonic blade.

40. The method of claim 39, further comprising:
calibrating, via the control circuit, a second signal generated by the sensor based on the fit curve; and
confirming, via the control circuit, that the sensor is generating accurate signals based on the calibration.

41. The method of claim 38 wherein adjusting the operating parameter is further based on waveform samples stored in a dynamically-updated lookup table.

42. An apparatus for controlling an end effector, the apparatus comprising:
a sensor configured to detect a signal in response to movement of a first tube relative to a second tube, the first tube driving movement of a clamp arm of the end effector;
a processor configured to:
determine a clamp arm position of the end effector relative to an ultrasonic blade of the end effector based on the signal;
determine a thickness of a tissue based on the clamp arm position;
determine an impedance of tissue in contact with the ultrasonic blade; and
determine a type of the tissue based on the impedance; and
a transducer configured to dynamically adjust a power output to the ultrasonic blade of the end effector during an ultrasonic treatment, to adjust a tissue cut based on the thickness and the type of the tissue.

43. The apparatus of claim 42, wherein the first tube is an inner tube and the second tube is an outer tube, the outer tube being moveable relative to the inner tube, the inner tube being static relative to the outer tube.

44. The apparatus of claim 42, wherein the first tube is an inner tube and the second tube is an outer tube, the inner tube being moveable relative to the outer tube, the outer tube being static relative to the inner tube.
